(19) United States
(12) Patent Application Publication
(10) Pub. No.: US 2020/0171319 A1
(43) Pub. Date: Jun. 4, 2020

(54) Structure Generating a Low Frequency Pulsed Electromagnetic Energy Field

(71) Applicant: Edmund Coffin, Ruckersville, VA (US)

(72) Inventor: Edmund Coffin, Ruckersville, VA (US)

(21) Appl. No.: 16/565,249

(22) Filed: Sep. 9, 2019

Related U.S. Application Data
(63) Continuation-in-part of application No. 15/877,346, filed on Jan. 22, 2018.
(60) Provisional application No. 62/728,164, filed on Sep. 7, 2018.
(60) Provisional application No. 62/448,716, filed on Jan. 20, 2017.

(51) Int. Cl. A61N 2/02; B68C 1/02; B32B 5/06; B32B 27/30; B32B 7/12; B32B 7/025; H01F 41/22; H01F 41/00

(52) U.S. Cl. CPC A61N 2/02; B68C 1/025; B32B 5/06; B32B 27/308; B68C 1/14; B32B 7/025; H01F 41/22; H01F 41/005; B32B 7/12

(57) ABSTRACT

An EM structure to emit a low frequency oscillating electromagnetic energy field has a nonpolar substrate, carbon fiber and an epoxy mixture to adhere the carbon fiber to a substrate, such as Kydex. The polarity changes from nonpolar to polar upon application of direct heat When the EM structure is configured with two opposing sides that have the same flex modulus, the EM structure is reactive to external materials. The electromagnetic field changes the structure, or energy level, of the unprocessed material to a positive, reinforcing energy while processed foods remain in a negative, draining state.

### FIELD OF THE INVENTION

This invention relates to an elongated arced structure, such as a saddle tree having a low frequency oscillating electromagnetic energy field generated by construction.

### BACKGROUND OF THE INVENTION

Electromagnetic fields, both from natural and man-made sources, are present everywhere in the environment. Frequency and wavelength characterize an electromagnetic field, which in an electromagnetic wave, are directly related to each other; the higher the frequency the shorter the wavelength.

Bioelectromagnetics, also known as bioelectromagnetism, is the study of the interaction between electromagnetic fields and biological entities. Areas of study include electrical or electromagnetic fields produced by living cells, tissues or organisms, including bioluminescent bacteria; for example, the cell membrane potential and the electric currents that flow in nerves and muscles, as a result of action potentials. Others include animal navigation utilizing the geomagnetic field; potential effects of man-made sources of electromagnetic fields like mobile phones; and developing new therapies to treat various conditions. The term can also refer to the ability of living cells, tissues, and organisms to produce electrical fields and the response of cells to electromagnetic fields. [Wikipedia]

Electromagnetic waves, through their electric and magnetic fields, can bring energy into a system, e.g. microwaves. Energy transfer is more efficient when the fields have the same frequency as the affected body.

The human nervous system generates electromagnetic fields, transmitting information in the form of electrical signals throughout our bodies. These signals affect nerves controlling internal functions of the body, along with our senses, muscle movement, and thinking. Technical information on electromagnetic fields and the effect on bodies is replete on the web.

Medical uses of low-intensity non-ionizing electromagnetic fields have also been explored in relationship to treatment of malaria and cancer. On Oct. 2, 2014 Chemical & Engineering News, ISSN 00009-2347, published Magnetic Fields Encourage Cellular Reprogramming, Bioengineering: Adult cells revert to pluripotent stem cells more efficiently in the presence of an applied magnetic field by Katherine Bourzac. Although some skepticism was expressed by other researchers, there were enough questions raised for some researchers to feel additional research is warranted.

There are studies showing a link between low frequency oscillating electromagnetic fields, their effect on water and the resulting health benefits due to improved hydration. It is generally know that life is predominantly water. Muscles are 75%; blood 82%, lungs 90%, brain 76% and bones 25% showing that water is critical to the quality of health. Dehydration affects the entire body, however the spine is especially affected as water fuels the hydraulic properties of the disc core. (Watercure by F. Batmanghelidj, M.D.). Further, according to Dr. Batmanghelidj “Fully 75 percent of the weight of the upper body is supported by the water volume that is stored in the disc core; 25 percent is supported by the fibrous materials around the disc.”

The benefits of cellular hydration are well known in the medical field as are the detriments of dehydration. Dehydration, rated as mild dehydration, less than 5% of the body's fluids; moderate dehydration, loss of 5-10% of body fluid; and severe dehydration, loss of 10-15% of body fluids which can result in a number of complications including It is believed by many that fully hydrated cells result in reduction of cell acidity, reduced autoimmune response, and increased immune system, to mention a few.

Although opinions vary regarding the cellular absorbability of plain water, it is generally agreed that fully hydrated cells are healthier than even mildly dehydrated cells. Fully hydrated cells more readily remove toxins and free radicals and generally improve cellular health.

The importance of cellular hydration is further discussed by Fisher, Dr. Howard, B.Sc., B.Ed., D.C. and Smirnov, Dr. Igor M. S., PhD. “Molecular Resonance Effect Technology: The Dynamic Effects on Human Physiology. Britannia Printers, Inc. ISBN 978-0-9780331-8-7. In this book they discuss the added benefit of water activated using Molecular Resonance Effect Technology (MRET). The device consists of pharmacologically active organic and inorganic substances that are exposed to a electromagnetic field and oscillating optical light. The frequency of the electromagnetic field is 7.8 and the light wavelength 600-700 nanometers. This exposure generates electromagnetic oscillations is similar to those found in healing waters throughout the world.

The book further states that “Consistent with the MRET theory, the applied electromagnetic field generates an excitation in the fractal geometry nano-ring of the polymer compound. Due to the phenomenon of piezoelectricity and intensive electrical activity of the fractal nano-rings, this polymer generates biologically active subtle electromagnetic oscillations. During the process of activation, the water is affected by specific patterns of subtle, low frequency, pro-biotic electromagnetic oscillations emitted by the MRET compound. The process of activation modifies the hydrogen-bonding patterns of water molecules and induces the formation of the long-range multilayer molecular structures compatible with the intercellular water structuring.”

An article published on Dr. Mercola's oneline health newsletter (mercola.com) stated that:

“Increase the structure of the water in your cells. Water is in every cell in your body, and this water is highly ordered (structured) and charged. If you don't have properly structured water in your cells, it can impact the functioning of the much larger protein molecules (and others) that interface with the cell. The water inside the cell also interfaces with water outside the cell, which has the opposite charge, creating a battery effect.

Your body's ability to generate electricity is actually a key part of your achieving health. Electrical charges delivered from cell to cell allows for nearly instantaneous communication within your body, and the messages conducted via these electrical signals are responsible for controlling the rhythm of your heartbeat, the movement of blood around your body and much more.

In fact, most of your biological processes are electrical. The water in your cells achieves its ordered structure from energy obtained from the environment, typically in the form of electromagnetic radiation, including sunlight and infrared heat.

But grounding may also play an important role. Just as water increases in structure when a negative charge is introduced by an electrode, the negatively charged electrons you receive when grounded help increase the structure of the water in your cells. By restructuring the water, you promote more efficient tissue healing. So, when you ground, you are charging every single cell in your body with energy your body can use for self-healing.”

An article published in December 2009 in the Indian Journal of Experimental Biology titled Low frequency pulsed electromagnetic field—A viable alternative therapy for arthritis by Kalaivani Ganesan et al. The article discusses the potential, based on clinical trials, of applying low frequency pulses electromagnetic fields, using inductive coupling, for the treatment of arthritis.

As research continues there is more evidence that physiological systems are affected by electromagnetic fields that are in harmony with the electrical systems of the body.

### SUMMARY OF THE INVENTION

The disclosed relates to the improvement in a structure to generate an electromagnetic field that can have an impact on water containing substances, including animate beings. Although the primary disclosure is for a saddle tree shaped element designed to produce an electromagnetic field for use with equines, it should be noted that it is the results achieved through the application of the electromagnetic wave that are critical. In a smaller configuration, the therapeutic structure can be used by a person with the same beneficial results. Other uses, such as bicycle seats, chair seats or backs, etc. are also possible. The disclosed structure is self-contained in that it creates an electromagnetic field without any outside element; however, it should be noted that in some applications it could be beneficial to add an external element to produce the electromagnetic field.

An electromagnetic structure to emit a low frequency oscillating electromagnetic energy field has a nonpolar substrate, carbon fiber, and an epoxy mixture to adhere the carbon fiber to a substrate, such as Kydex. The chemical make up of the substrate is such that its polarity changes from nonpolar to polar upon application of direct heat. Prior to application of direct heat the substrate is sanded in a cross hatch pattern, washed, and then dried completely. Once completely dry, the direct heat is applied, generally through flaming with the blue portion of a propane torch flame in contact with the substrate. The flaming brings the substrate temperature up to approximately 150-180 degrees F. The carbon fiber is immersed in the mixture of soft and hard epoxies until saturated and, after removal of the excess epoxy, placed on the substrate. During the application of the carbon fiber, the temperature of the substrate should not drop below 50% of the direct heat temperature and preferably no lower than 100 degrees F. The EM structure is then dried.

When the EM structure is configured with two opposing sides that have the same flex modulus, the EM structure is reactive to external materials. Materials that are unprocessed, e.g. well water, fruit, vegetables, will strengthen the structure and make the side proximate the material more difficult to flex. In contrast processed materials, such as beer, soda, foods containing chemical, when placed proximate the structure will make the side substantially easier to flex. Unprocessed foods placed proximate one side of the EM structure will not affect the flex initially but will, after about 30 minutes proximity to the structure, make the side to which it is proximate more difficult to move. The electromagnetic field changes the structure, or energy level, of the unprocessed material to a positive, reinforcing energy while processed foods remain in a negative, draining state.

### BRIEF DESCRIPTION OF THE DRAWINGS

The advantages of the instant disclosure will become more apparent when read with the specification and the drawings, wherein:

FIG. 1 shows oscilloscope readings from a saddle tree at rest, in accordance with the invention;

FIG. 2 shows oscilloscope readings from a saddle tree flexed with approximately 15 pounds of pressure, in accordance with the invention;

FIG. 3 shows oscilloscope readings from a saddle tree flexed with approximately 30 pounds of pressure, in accordance with the invention;

FIG. 4 shows oscilloscope readings from a saddle tree flexed with approximately 45 pounds of pressure, in accordance with the invention;

FIG. 5 shows oscilloscope readings from a saddle tree flexed with approximately 60 pounds of pressure, in accordance with the invention;

FIG. 6 shows oscilloscope readings from a saddle tree flexed with approximately 75 pounds of pressure, in accordance with the invention;

FIG. 7 shows oscilloscope readings from a saddle tree flexed with approximately 90 pounds of pressure, in accordance with the invention;

FIG. 8 shows oscilloscope readings from a saddle tress flexed with approximately 105 pounds of pressure, in accordance with the invention;

FIG. 9 show the top of a therapy tree without covering or cut central channel, in accordance with the invention;

FIG. 10 show the underside of the therapy tree of FIG. 9 without covering or cut central channel, in accordance with the invention;

FIG. 11 is a view of the underside of the therapy tree covered in carbon fiber with the central channel cut, in accordance with the invention;

FIG. 12 is a view of the top of the therapy tree of FIG. 11 with the addition of strap, in accordance with the invention;

FIG. 13 is a top view of the therapy tree of FIG. 12 with the girth and leather covering, in accordance with the invention; and

FIG. 14 is a figure of the therapy tree of FIG. 13 on a horse, in accordance with the invention.

FIG. 15 shows the underside of a saddletree having carbon fiber covering and copper wire leads in accordance with the invention;

FIG. 16 is a graph from a data logging meter of the saddletree of FIG. 1 in accordance with the invention;

FIG. 17 shows the underside of a saddletree having carbon fiber covering and copper wire leads with a second field in proximity to the wire leads in accordance with the invention;

FIG. 18 is a graph from a data logging meter of the saddletree of FIG. 2 showing the change in the electromagnetic field upon introduction of a second field in accordance with the invention;

FIG. 19 is a graph representing the electromagnetic field when placed on a horse in accordance with the invention; and

FIG. 20 is a graph representing the electromagnetic field when placed on a horse in accordance with the invention:

FIG. 21 is a graph illustrating the millivolt reading upon placement the saddle tree on a horse at rest; and

In FIG. 22 is a graph illustrating the millivolt reading after one day of placement of the saddletree on a horse at rest.

### DETAILED DESCRIPTION OF THE INVENTION

Definitions

For the purposes as employed herein the term “aquaporin” shall refer to the integral membrane proteins that form pores in the membrane of biological cells, mainly facilitating transport of water between cells. (Wickipedia) In 2003, the Nobel Prize for Chemistry was awarded for the explanation of how Aquaporins' (the cells water channels) carry water, one molecule at a time through the cell membranes.

For the purposes as employed herein, the term “base output” shall refer millivolt output by the disclosed electromagnetic (EM) structure without external influence by an object or human presence.

For the purposes as employed herein, the term “affected output” shall refer to millivolt output by the disclosed EM structure with external influence by an object or human presence.

For the purposes as employed herein, the term “applied kinesiology” shall refer to the non-invasive diagnostic system using muscle testing as a tool for evaluating neurological function.

For the purposes as employed herein, the term “converted water” shall refer to water that has been exposed to an electromagnetic field.

For the purposes as employed herein the term “electromagnetic” shall refer to the interrelation of electric currents or fields and magnetic fields.

For the purposes as employed herein the term “electromagnetic field” shall refer to a field of force that consists of both electric and magnetic components, resulting from the motion of an electric charge and containing a definite amount of electromagnetic energy.

For the purposes as employed herein the term “electromagnetic induction” shall refer to the production of an electromotive force (i.e., voltage) across an electrical conductor due to its dynamic interaction with a magnetic field.

For the purposes as employed herein, the term “substrate” shall refer to a material, natural or synthetic, that is used as the base or body of the object and that, in combination with the method disclosed herein, produces electromagnetic fields. The substrate used is manufactured from a material such as Kydex, which is a mixture of polyvinyl chloride, chlorinated polyvinyl chloride, acrylic polymer, as well as a mixture of processing aids, impact modifiers, heat stabilizers and lubricants and an organotin compound. The organotin compound and processing aid, impact modifier, heat stabilizer, lubricant and pigments mixtures are proprietary to Sekisui SPI and protected as trade secrets. Kydex further has stain resistant properties. Other substrate materials can be used, however they must have the characteristics of Kydex to produce optimum results. Due to the number of elements within the Kydex and the proprietary information of some ingredients, it is unknown whether the substitution of another material will produce the same results.

For the purposes as employed herein the term “EM structure” shall refer to a substrate that has been prepared in accordance with the disclosure hereinafter, including the carbon fiber and epoxy and that emits a low frequency, oscillating electromagnetic energy field.

For the purposes as employed herein the term “flex” shall mean to bend by expansion of one surface and contraction of the opposing surface.

For the purposes as employed herein the term “resonance” shall mean the condition of a system in which there is a sharp maximum probability for the absorption of electromagnetic radiation or capture of particles. Resonance is a phenomenon that occurs when a vibrating system or external force drives another system to oscillate with greater amplitude at a specific preferential frequency.

For the purposes as employed herein the term “flex modulus” shall refer to the physical property denoting the ability for a material to bend. An increased or strengthened flex modulus refers to a decrease in flex (or an increased resistance to flex). A decreased or weakened flex modulus refers to an increase in flex (or a decreased resistance to flex).

For the purposes as employed herein, the term “epoxy” shall refer to any adhesive, soft or hard, applicable to use with the chosen composite and substrate. These include various solid or semisolid amorphous fusible natural organic substances as well as any of a large class of synthetic products that have some of the physical properties of natural adhesives but are different chemically and are used chiefly in plastics.

For the purposes as employed herein, the term “soft epoxy” shall refer to any adhesive applicable for use with the chosen substrate that has an elasticity of about 150,000 PSI thereby being more flexible than standard epoxies while stiffer than adhesive sealants. The softer epoxy should have the ability to make structural bonds that can absorb the stress of expansion, contraction, shock, and vibration. It is ideal for bonding dissimilar materials.

For the purposes as employed herein, the term “hard epoxy” shall refer to any adhesive applicable for use with the chosen substrate that has strong physical properties for structural bonding.

For the purposes as employed herein, the term “polar” refers to a material having molecules with a positive electrical charge on one side and a negative electrical charge on the other side. [thoughtco.com]

For the purposes as employed herein, the term “magnetic field homogeneity” refers to a uniform electric field which has the same strength and direction at each point.

For the purposes as employed herein, the term “non polar” refers to a material having molecules which have no separation of charge, so no positive or negative poles are formed. In other words, the electrical charges of nonpolar molecules are evenly distributed across the molecule. Nonpolar molecules tend to dissolve well in nonpolar solvents, which are frequently organic solvents. [thoughtco.com]

For the purposes as employed herein, the term “therapeutic structure” shall refer to a structure that through its design and configuration will produce an electromagnetic field.

For the purposes as employed herein the term “capacitive coupling” shall refer to the application of low frequency pulsed electromagnetic fields (PEMF) using electrodes placed directly on the skin surrounding the area of interest.

For the purposes as employed herein the term “inductive coupling” shall refer to the application of low frequency pulsed electromagnetic fields (PEMF) induces an electric field that products a current in the body's connective tissue.

The disclosed relates to the improvement in a structure to generate an electromagnetic field that can have an impact on water containing substances, including animate beings. Although the primary disclosure is for a saddle tree shaped element designed to produce an electromagnetic field for use with equines, it should be noted that it is the results achieved through the application of the electromagnetic wave that are critical. In a smaller configuration, the therapeutic structure can be used by a person with the same beneficial results. Other uses, such as bicycle seats, chair seats or backs, etc. are also possible. The disclosed structure is self-contained in that it creates an electromagnetic field without any outside element; however, it should be noted that in some applications it could be beneficial to add an external element to produce the electromagnetic field.

The applicant has a long history of working to improve saddle trees to optimize the comfort for a horse as well as the connection between a horse and rider. Further, the applicant trains and evaluates performance horses for their owners as well as owns a number of horses. Through the work to design and manufacture the optimal saddle, it was found that with a new manufacturing process changes were noted in horses both when being saddled in preparation for being ridden and when being exercised in the saddle. The saddle has a therapeutic effect on a horse. In an evaluation from to Melissa Holland, DVM, DACVA, she states “When placed on a horse's back for 20 minutes with the girth on, the technology has consistently demonstrated an ability to significantly reduce or completely eradicate back pain and create a state of deep relaxation. This effect is due, in part, to an electromagnetic phenomenon in the tree that is a result of the combination of the materials used, the manufacturing processes, and the geometric features of the tree itself.

There are a number of short term and long-term positive effects produced in a horse by the regular use of saddles containing this technology. These include, but may not be limited to:

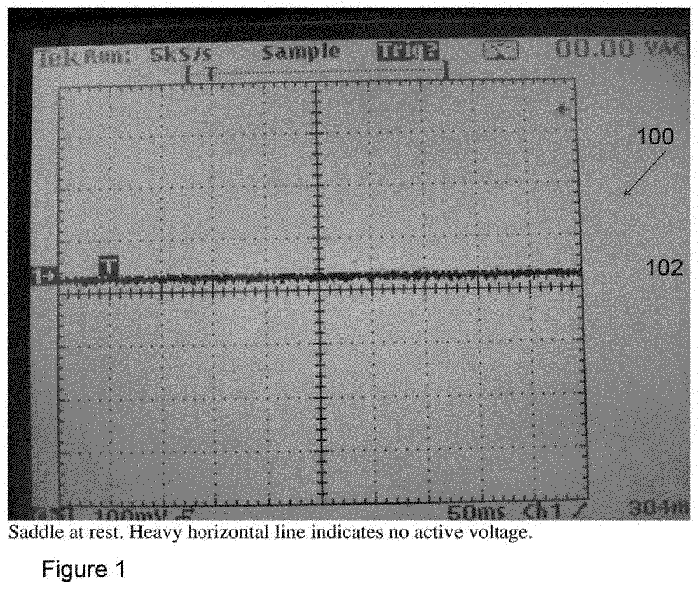

Relief of muscular pain, most especially along the horse's back muscles—longissimus and the overlying and contiguous latissimus dorsi, FIG. 1. These muscles are highly integrated with the other major muscle groups of the topline and the hindquarter. Tension and toxin build-up in these two muscle groups can propagate to the other major muscle groups of the hindquarter and through the neck, creating stiffness, resistance, and lack of fluid, forward motion. These muscles also attach to the processes of the spinal vertebra. Muscle dysfunction or fatigue from misuse or overuse changes normal muscle length which may ‘pull’ the vertebrae ‘out of alignment’ and reduce range of motion. Massage and chiropractic adjustment may not produce expected results or results which do not last. Note, the saddle and the rider sit along the longissimus and the latissimus.

Improvement in cardiovascular function and faster recovery from exercise.

Improvement in immune system function, including reductions in allergic skin reactions and improvement in chronic allergic conditions.

Improvement in hoof condition.

Reduction in apprehension in general and during exercise/work, shoeing, veterinary care, etc.

Reduction in over-reaction to adverse stimuli such as new things in the routine or environment or to new challenges in training or work, such as a new jump in the ring or something ‘spooky’ on the trail.

Improved attention and focus during work and reduced resistance to learning during work.”

The first change was that the horses all exhibited a greater sense of relaxation. Next was noted significant reduction, or elimination of back pain in a 20-30 minute period just while standing. In the Fall of 2015, the applicant began an initiative to have all test horses wear saddles at rest for at least 40 minutes per day, 7 days per week for the purpose of exploring the long term benefits of the electromagnetic field generated by the saddle. Soon thereafter, another physiological change was noted—improved cardiovascular functioning, including lower resting heart rate and recovery after exercise when using a saddle having the tree manufactured in the disclosed manner. Then over time greater health benefits on a broad spectrum of physiological functions were noted. These benefits included improved digestion, food utilization, elimination of digestive disorders, improved lymphatic circulation, elimination of generic swelling, improved immune system, reduction or elimination of allergies, and notable improvements to hydration levels. As the common denominator of these physiological functions is water, the hypothesis was the electromagnetic field was somehow creating improved levels of intracellular water.

Horses in training that were switched from prior art saddles to the saddles containing the saddle tree disclosed herein showed a marked improvement in movement and a lessening of resistance to being saddled. They demonstrated improved balance, less resistance to riders' requests, longer strides, improved straightness and ability to bend in either direction, better carriage, and improved jumping style.

Seeking an explanation of the favorable reaction by the horses, it was found that some saddle trees did not flex symmetrically after periods of use. This was discovered as there was an immediate and precipitous decline in the performance of test horses, sometimes even when wearing a saddle at rest. Upon close examination, small surface anomalies were discovered that, when eliminated, restored the symmetrical flexion. These anomalies were always of a certain nature, always relating to the consistency of the carbon fiber surface and consistent in their effect on proper flexion. When the saddles were returned to symmetrical flexion, the performance of the test horses, even at rest, was immediately restored. It was hypothesized that a current or energy flow was occurring on the saddle tree surface and that surface anomalies were disrupting the flow. This disruption in the energy flow was triggering a negative response from horses. It was then discovered that deliberately caused anomalies could have predictable consequences on the symmetry of saddle flexibility and horse response. In the many thousands of tests conducted over the course of several years, this relationship between surface anomalies, asymmetry of flexion, and negative horse response has been completely consistent.

Since the original configuration was a saddle tree, the further experiments were also conducted using the prepared saddle trees. This was not only for the consistency of the test model but for the ability to easily flex the saddle tree to test for the therapeutic benefits of the structure. As disclosed hereinafter a therapeutic structure, the size of a bicycle seat, was also used for testing, however due to the size, it was difficult to test the degree of flex. The size reduction did however provide that the field existed in other configurations and that the size of the field is directly related to the size of the EM structure. For example, a therapeutic EM structure the size of a saddle tree emits a field approximately six foot while a therapeutic EM structure the size of a bicycle seat emits a field approximately 3 feet, extending in all directions forming a cube. There is no known reason why the electromagnetic field would not work on a flat EM structure the same as it does on the curved saddle tree.

In order to determine whether voltage was being generated through flex of the tree and if so, how much and how did it change, a test device was built. A brace held the saddle tree in position, permitting flex but no other movement. A pneumatic powered pressure bar was placed to be in the same position as would be a rider to place pressure on the seat of the saddle. A Tektronix® digital oscilloscope was used to measure surface voltage versus time. The probes of the oscilloscope were surface mounted to the top and bottom of the saddle tree and remained in the same position throughout the testing. The scaling of voltage and time were also held constant to enable the graphs of FIGS. 1-8 to be compared without re-scaling.

The graphs illustrated in FIGS. 1-8 reflect the screen read outs during the applications of identical pressure from the pneumatics. The oscilloscope produces a chart of voltage as a function of time with the greater the deviation from the horizontal centerline, the greater the voltage. The time is represented as moving left to right with the oldest time on the left.

The saddle tree was subjected to a series of flexing motions, cantle to pommel. The duration of the saddle flex was based on the approximate time it takes a rider to complete one post. To consistently track the changes, each flexing cycle was increased by approximately 15 pounds from the previous cycle with the digital record being photographed.

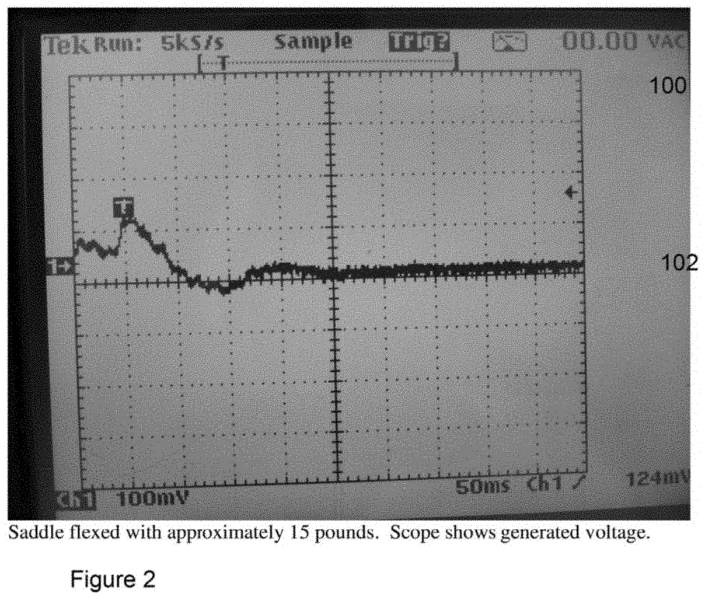

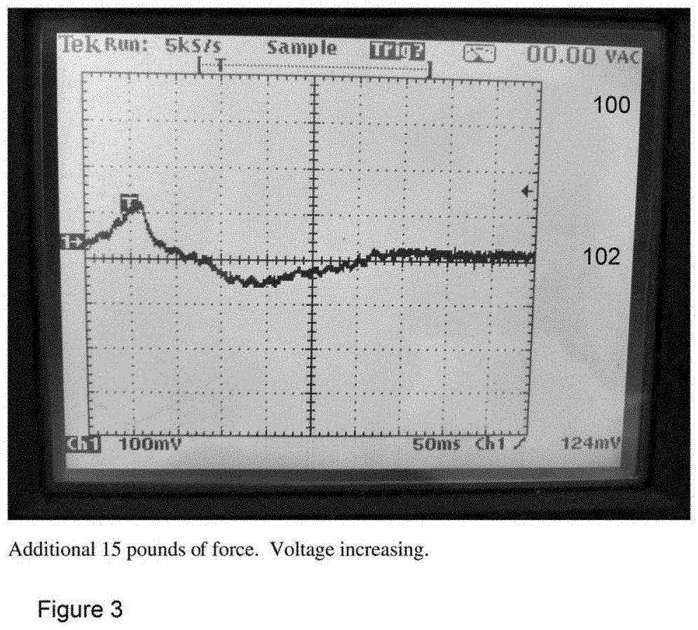

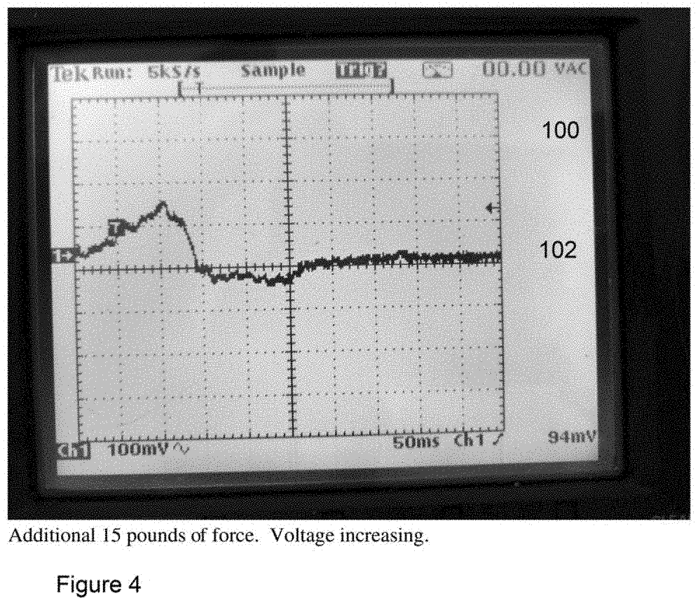

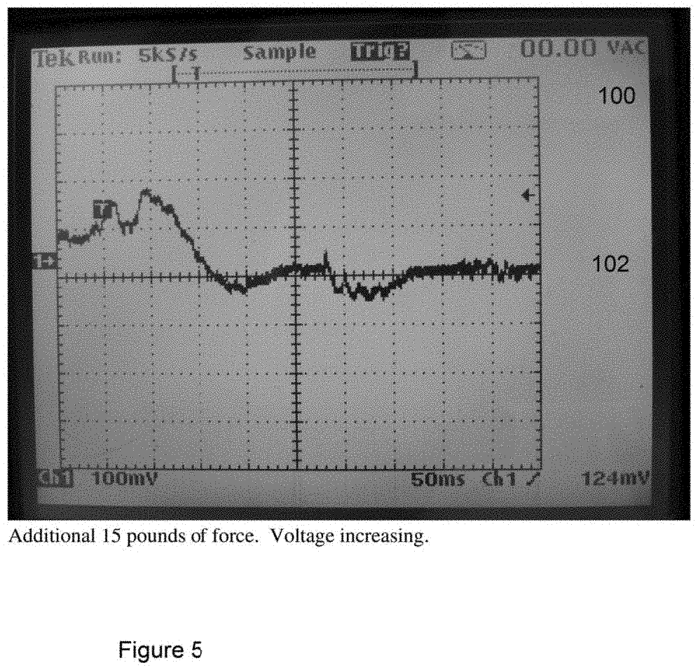

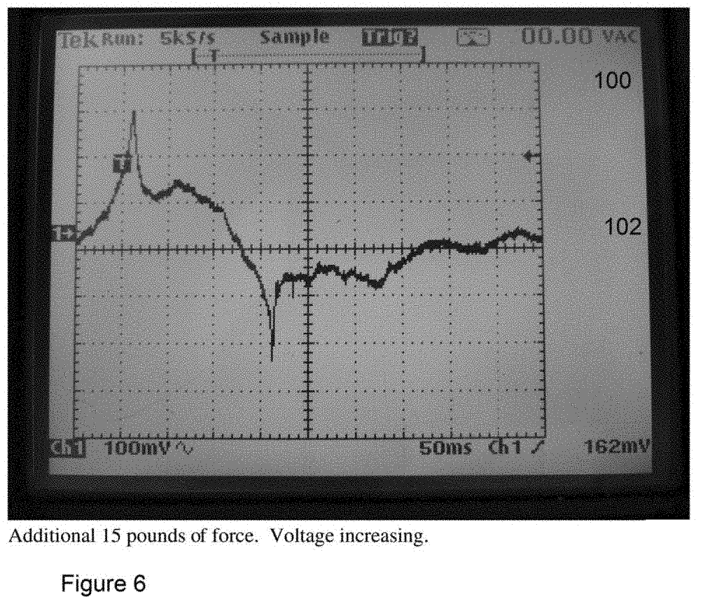

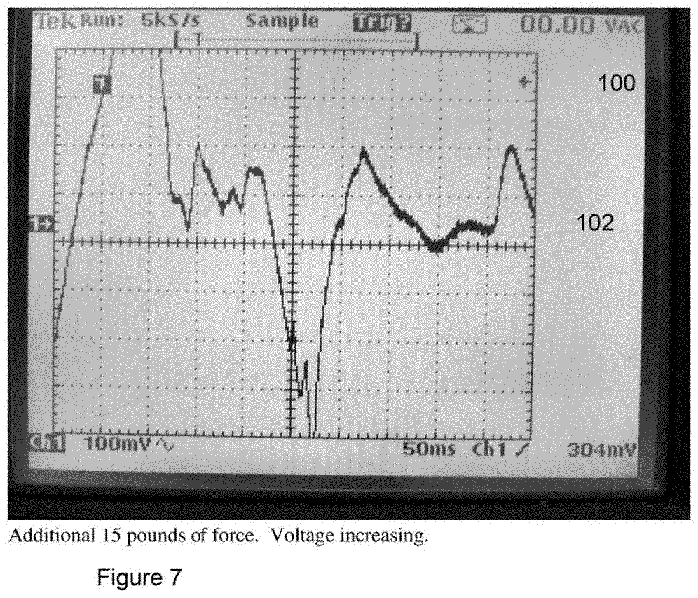

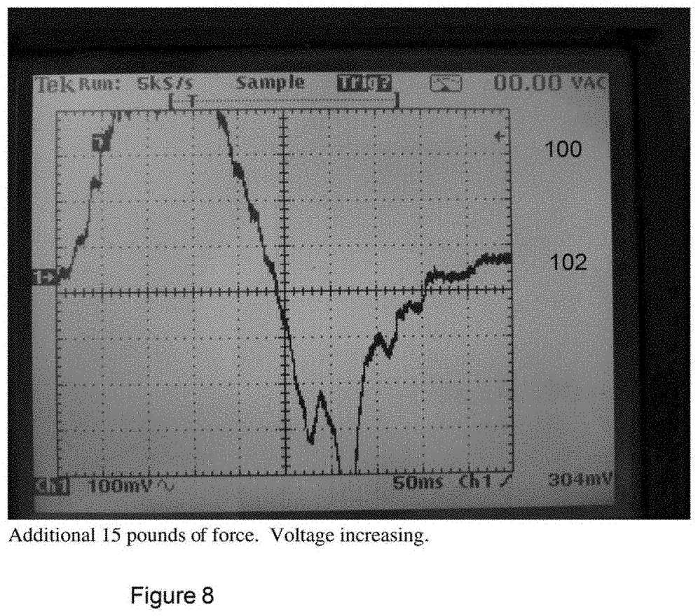

The graph 100 of FIGS. 1-8 illustrate the voltage changes through the pressure addition cycles. In FIG. 1, as indicated by the heavy horizontal voltage line 102, the saddle is non-flexed and in a resting state. In FIG. 2 the pneumatic device was set to apply 15 pounds of pressure to the saddle, initiating a voltage increase as indicated by the voltage line 102. In FIG. 3, the pressure is increased to 30 pounds with the voltage, as indicated by voltage line 102, increasing. In FIG. 4 the pressure is increased to 45 pounds as shown by the voltage line 102. FIG. 5 illustrates the voltage increase in voltage line 102 when the pressure is increased to 60 pounds. In FIG. 6 the pressure is at 75 pounds and there is a substantial change in the voltage line 102. The voltage line 102 changes dramatically in FIG. 7 with the application of 90 pounds of pressure and, in FIG. 8, with 105 pounds of pressure, the voltage line 102 shows further increase.

As can be seen from FIG. 1, energy inherently exists, even when the saddle is at rest, and increases with flex, verifying that potential energy is being created by movement of the saddle. It has been found that many items containing water, either within their structure (human body) or contained within an inert container (glass of juice), are affected by the electromagnetic waves from the saddle. In order to test the effects of the electromagnetic fields on objects applied kinesiology is used. Holding a container of converted water to a user's chest, a user cannot be tipped over, a raised leg or arm cannot be pressed back into position. The increase can be substantial, however it is difficult to determine an actual percentage of increase although the estimation is that it can be up to 500%.

To further explore the effect of PEMFs, distilled water was used as an energy barrier. The distilled water does not contain the quantity of ions found in regular water and it is believed that it is the lack of ions and minerals is the reason that the electromagnetic fields are repelled by the distilled water.

When a container of water is placed in the vicinity of the saddle, the water absorbs the 3000 cm infra-red spectrum, altering the structure of the water. It is believed that the charge within the water wants to obtain the strongest state which, for water, is a negative electrical charge.

As found in testing at Ecole Polytechnique Federate de Lausanne by R Scheu et al and published in Phys.org on Jul. 16, 2014 by Sarah Perrin: “The scientists' observations showed that the hydrogen bonds—that is to say, the electrostatic force binding the hydrogen atoms with others such as oxygen or nitrogen—were more than 6 times more abundant when the ions were negatively charged. The hydrogen bonds were also much stronger. Somehow, water maintains more collaborative relationships with such negative ions. Thus, they get much more hydrated and their effects, in particular on the orientation and alignment of water molecules at the interface between the two substances, were stronger and more stable.” Read more at: https://phys.org/news/2014-07-molecules-favor-negative.html#jCp

While the energy of the tree wants to dissipate, the tree also wants to receive. If the energy received is compatible, a resonance frequency is developed and the tree strengthened, making it more difficult to flex. When the saddle tree is in resonance with the surrounding items, whether it is a horse or a glass of water, both reach their maximum strength. Within approximately 20 to 30 minutes the resonance takes effect, creating converted water.

Not all items resonate with the energy frequency of the saddle tree. When distilled water is placed in the vicinity of one side of a saddle, the PEMF's have nothing within which to be absorbed with in the water. The energy then points out the weak spot in the saddle through the weakening of the flex on that side. When a glass of distilled water is place on either side of the saddle, both sides will show weakness by requiring less pressure required to flex.

The hypothesis is that the piezo electric phenomena creates voltage that is stored within the components of the saddle tree, causing it to act like a capacitor. During rest the voltage registered is similar to a leaky battery. It is unknown yet how much time it would take to drain the saddle as a 2.5 year old saddle tree produced the same output as a newly constructed saddle tree.

As the saddle works as an antenna, emitting a certain frequency, the configuration of the saddle is critical. The saddle tree tested is constructed in accordance with U.S. Pat. No. 9,586,809 and U.S. Ser. No. 15/877,346, both of which are incorporated herein as through recited in full. It should be noted, however that although the foregoing provide information on the construction of the tree, the trees disclosed within the incorporated teachings are designed to support the weight of a person. Therefore, although the construction of the Kydex is the same, the reinforcing and concern with placement of elements is not necessarily applicable with the therapy tree.

The waves along the edge of the tree serve as antenna and serve to direct the energy stored in the tree outward. In testing it has been found that the energy travels in the direction of the carbon fiber covering the substrate. One test used a piece of tape that when aligned with the carbon fiber produced a negative effect in the flexibility of the saddle tree. However, when the tape was rotated to 45 degrees, the negative effect was eliminated.

The electromagnetic field around the saddle tree extends along the sides of the tree, coinciding with the waves as described in the aforenoted application. The fields merge in the area of the cantle and pommel, and an item placed in the front or back of the saddle has no effect on either the item or the saddle tree.

In an experiment taking 100 mL of the converted water and 100 mL of tap water, approximately two-tenths of the converted water was added to the tap water. The mixed tap water was tested, using applied kinesiology, and produced the same results as the converted water.

A container of tap water was placed next to the saddle tree and, within approximately 30 minutes was removed and checked for conversion using applied kinesiology. The container of converted water was placed, out of electromagnetic field range of the saddle tree, close to a container of tap water. In approximately 20 minutes the tap water was tested and found to be converted.

An egg was placed within the electromagnetic field of the saddle tree and, after approximately 20 minutes, the egg was removed from the electromagnetic field. The egg was tested using applied kinesiology and found to be converted in the same manner as the water. The egg was marked to indicate that it had been converted.

The marked converted egg was placed, outside the electromagnetic field of the saddle tree, proximate an unconverted egg. The two eggs were left for approximately 30 minutes and the unmarked egg removed from the electromagnetic field of the converted egg. The unmarked egg was tested and found to possess the same qualities as the converted egg.

In light of the foregoing, the therapy tree, based on the saddle tree design, was developed. The therapy tree 200 is manufactured from, and in the same manner, as the saddle tree disclosed in detail in U.S. Pat. No. 9,586,809 and co-pending application Ser. No. 15/482,433 which is incorporated herein as though recited in full.

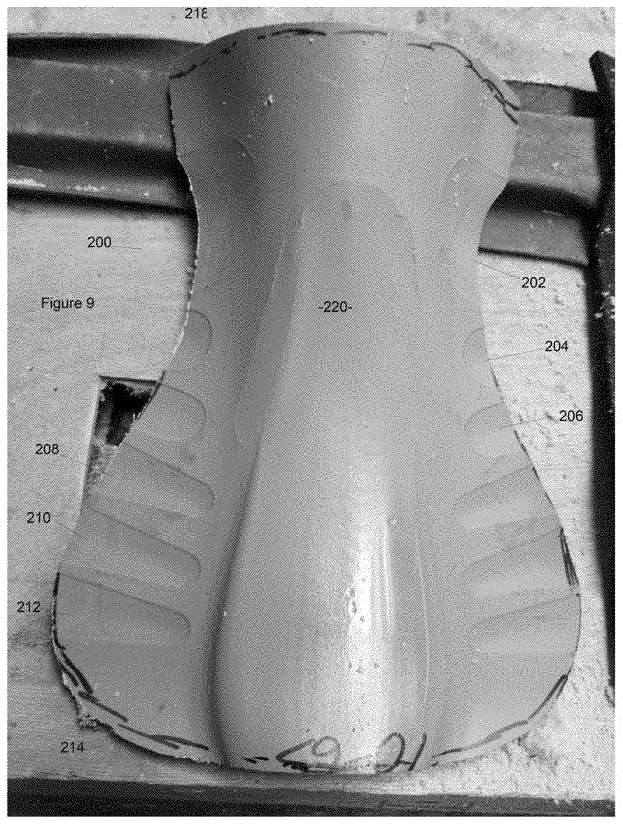

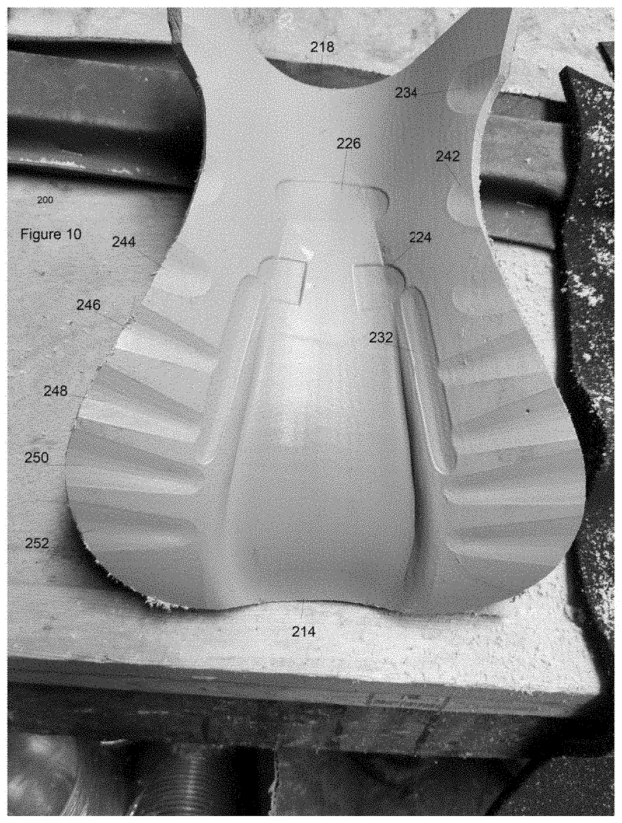

In FIGS. 9 and 10 the therapy tree 200 is illustrated in the unmolded form prior to the cutting of the center channel 220 or addition of carbon fiber. The presence of the wave depressions and center channel are critical to the creation of the electromagnetic field. Although the detailed construction is incorporated herein through the incorporation of the above applications, a description of the therapy tree is disclosed herein.

Side wave depressions 234, 242, 246, 246, 248, 250 and 252 are molded into the bottom 214 of the saddle tree 200, and wave depressions 202, 204, 206, 208, 210, and 212 which are off set from depressions 234, 242, 246, 246, 248, 250 and 252, are molded into the top, as shown also in FIGS. 9 and 10. Waves 244 and 206 have a half oval configuration while waves 208-212, and their counterparts 246-252 are cone shaped. The waves are about 0.03 ( 1/32) of an inch deep and spaced along the edge of each side of the tree 200. The offset wave depressions form an undulating affect that contributes to creation of the electromagnetic field.

The designation of top or bottom waves is for example and the waves can be reversed with the first wave being on the top surface. The critical feature is the placement and dimensioning of the waves.

The depth of the wave depressions 202, 204, 206, 208, 210, 212, 234, 242, 246, 246, 248, 250 and 252 directly affect the performance of the therapy tree, or other structure, as the greater the depth, the greater the amount of flex.

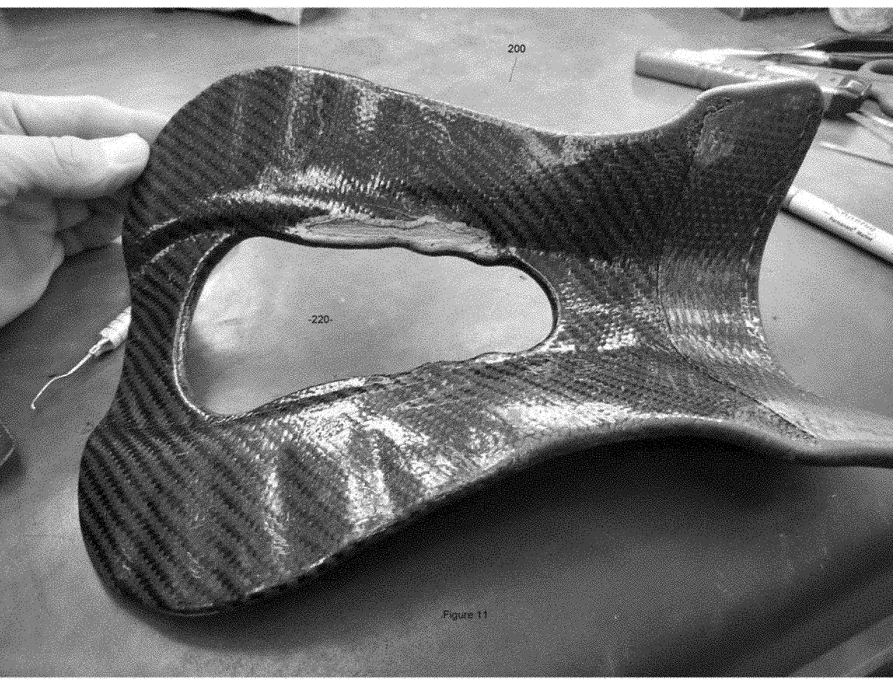

Between the channel 220 (FIG. 11) and the pommel 218, on the underside of the tree 200, is the pommel wave 226. As physics requires that a wave needs an edge to flex, the wave 71 has either the ellipse 72 and slots 73a and 73b, or the slots 574 and 576 to provide the necessary edge. The pommel wave 71 enables the torque created by movement of the horse to “move through” the saddle in a controlled manner without resistance or obstruction.

In FIG. 11 the central channel 220 has been cut out and the therapy tree 200 covered with carbon fiber and adhered with an epoxy as taught in the foregoing application and patent. The central channel 220 is critical as it divides the electromagnetic field, thereby permitting one side to flex more or less than the opposing side.

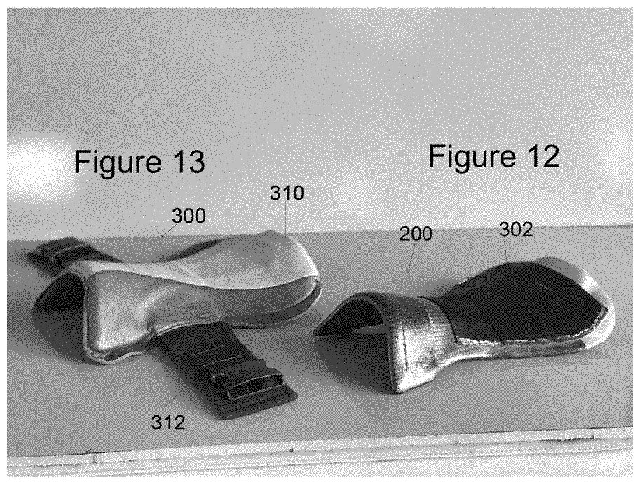

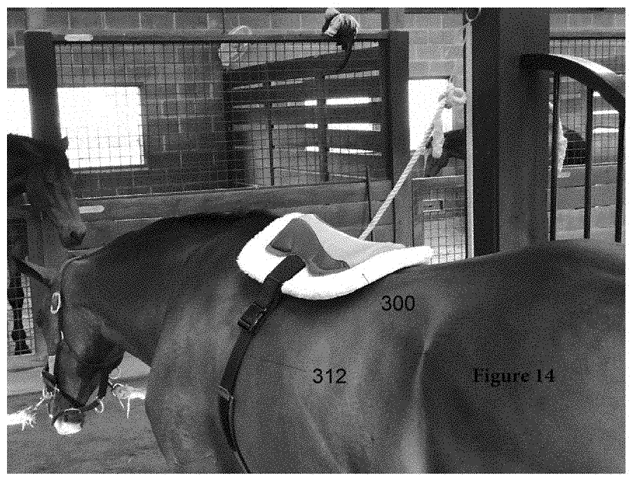

In FIG. 12 carbon fiber strips 302 have been secured to the tree 200, covering the central channel 220. In FIG. 13 the girth 312 has been added and the tree 200 covered with a leather covering 310 thereby completing the tree 300. The leather covering 310 serves to focus the electromagnetic field.

In FIG. 14 the complete tree 300 is placed on a horse and secured by the girth 312.

Materials and Method of Construction

The substrate used is a nonpolar material that does not have normal bonding processes and requires special preparation to adhere the carbon fiber. It is believed that the process required to prepare the substrate for adhesion is integral to the creation of the electromagnetic field.

The substrate is initially prepped by sanding with 150-220 grit in a cross hatched pattern. This pattern etches the surface and increases the mechanical bond between the substrate and carbon fiber. Although the sanding does not need to be absolutely perfect, any areas not sanded are subject to delamination.

Once sanded the substrate is washed and placed in a heating element producing dry heat at 120 degrees. The substrate is a porous material and absorbs moisture during the washing process. In order to provide adherence, the substrate must be moisture free. The amount of time required to dry the substrate is dependent upon the thickness and size. For example a ⅜ inch thick substrate would be dried in a heating element for about 12 hours while a ⅛ inch thick substrate would be dried for approximately 6 hours. Ambient drying is possible but timing is dependent upon the current weather conditions.

Once dry, a propane torch is then run over the entire surface sufficient close to produce a blue flame on the surface. This is a critical step as the flaming changes the polarity of the substrate. During the flaming, the temperature is raised to about 160 to 180 degrees and the surface ionized, thereby trading electrons and changing the polarity. Care must be taken not to burn or melt the substrate while still raising the temperature to the degree where the polarity is changed.

As the substrate does not hold heat, the epoxy saturated carbon fiber must begin to be applied with one or two minutes. Therefore it is preferable that the carbon fiber be prepared and waiting at the time of commencing the flaming. During the carbon fiber application process the substrate should not fall much below 100 degrees or at most 50% of the post flaming temperature. In order to successfully avoid an undesired drop in temperature and to avoid delamination, all materials, including air, equipment, chemicals and surfaces must be maintained at between 70-80 degrees. A dedicated room maintained at the desired temperature and containing all components is recommended to prevent delamination.

The resin preparation used to adhere the carbon fiber to the substrate is a mixture of hard and soft epoxies. An example of a hard epoxy would be West Systems 105/205 mix or the equivalent. West Systems' G/flex two part adhesive, or its equivalent, is used as the soft epoxy. A mixture is formed of 1 part hard epoxy and 1 parts soft epoxy. Can be altered depending on end use and desired flex difference.

The carbon fiber is placed within the epoxy mixture until it reaches the point of complete saturation, the point at which no more epoxy can be absorbed. At this point it is squeegeed to remove excess epoxy and then placed on the still hot substrate. When the EM substrate is a saddle tree, it is placed on the underside of the tree.

The coated substrate is baked at 105 degrees to cure for approximately 4 hours. Alternatively, the substrate can be left at room temperature for curing, with the time difference between baking and ambient curing being based on the room temperature.

The result of the foregoing process produces an EM structure having piezo electric, phenomena producing low frequency oscillating electromagnetic properties in the 7.8 range, closely aligned with the levels naturally produced by the human body. As explained by Lipkova, J. and Cechak, J in “Human electromagnetic emission in the ELF band”. Measurement Science Review, Volume 5, Section 2, 2005. Further “EEG has shown that waves do not expand to the brain only, but through the whole nervous system (through the perineural system) to every part of the organism.” Although at high levels, such as microwaves, electromagnetic waves are dangers, when aligned with the waves naturally produced by the body, they can provide a number of benefits.

The original testing to determine the existence of the field is described hereinafter.

Testing Example I—Discovery of Milivolts and Electromagnetic Field

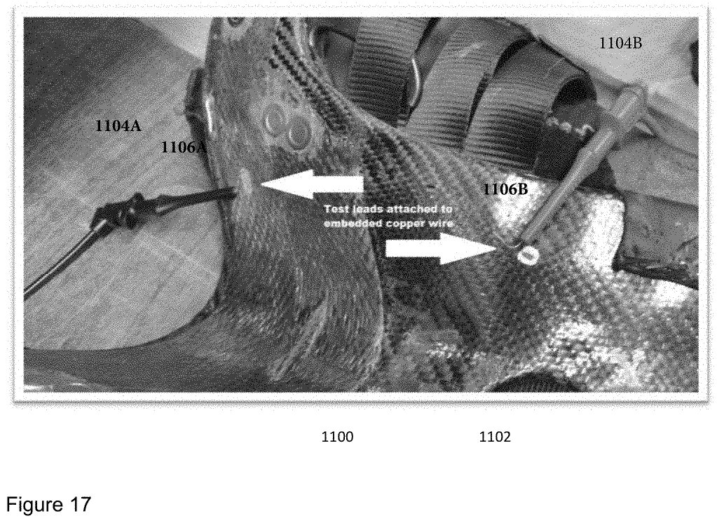

In FIG. 17 the underside of a substrate with an adhesive resin/carbon fiber 1102 coated saddletree 1100 is illustrated. The saddletree 1100 has two (2) layers of adhesive resin coated carbon fiber 1102 covering the substrate. Copper wires 1106A and 1106B have been placed within the resin/carbon fiber 1102 without coming into contact with the substrate. Test probes, negative probe 1104A and positive probe 11046 are then attached to the copper wires 1106A and 11066. The test probes 104A and 104B are connected to a data logging meter, such as a multimeter.

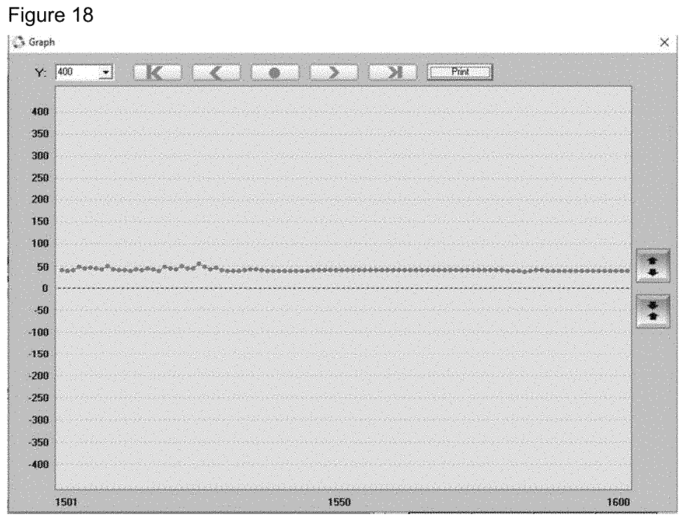

Readings from the probes 1104A and 11046, as received from the copper wires 1106A and 11066, are read by the data logging meter. The graph of FIG. 18 shows the reading from the data logging meter with data points at one second intervals, providing a baseline millivolt reading of 50.

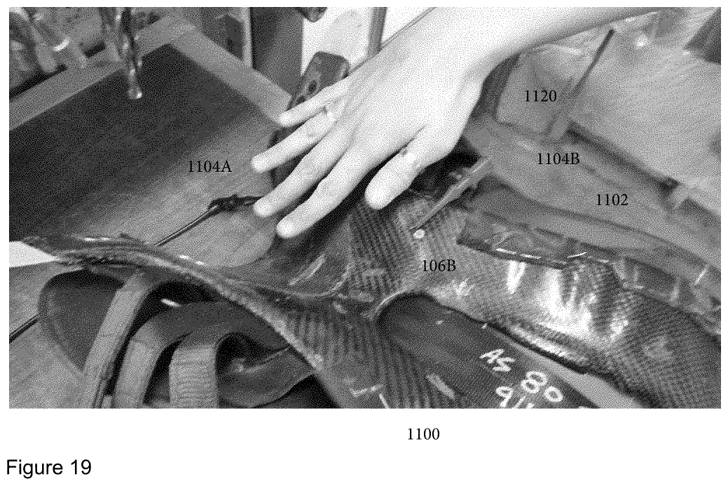

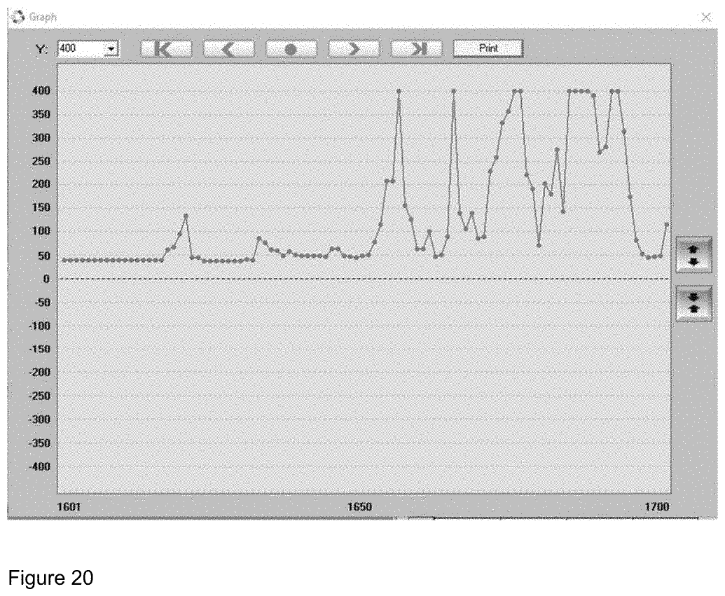

Without moving the probes, a second field 1120 (in this illustration a hand) is introduced proximate to, but not touching, the carbon fiber 1102 as illustrated in FIG. 19. The graph of FIG. 20 illustrates the data points as the source of the second field 1120 is moved around the saddletree 1100. As can be seen from the graph of FIG. 20, the baseline reading of 50 increases to 400 as the second field approaches and drops back to 50 as the second field is removed. Moving the second field closer and further from the saddletree raises and lowers the millivolt readings.

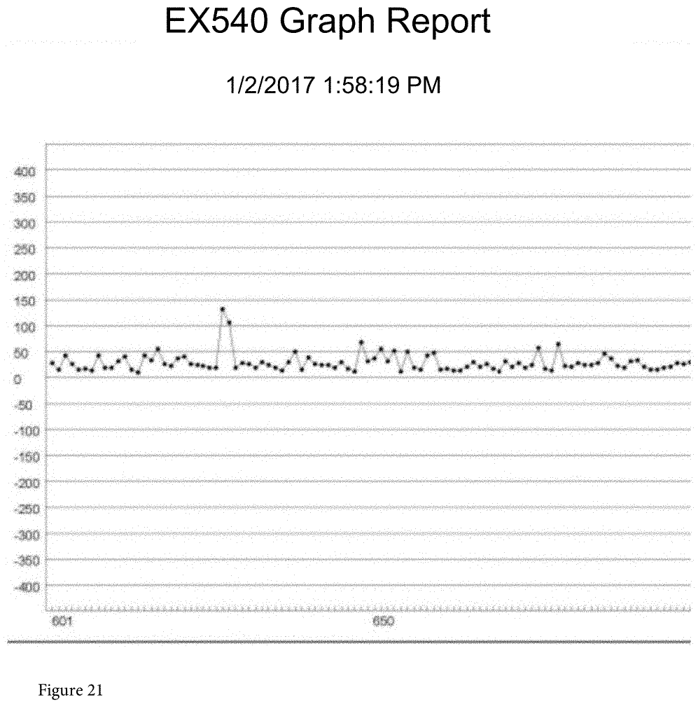

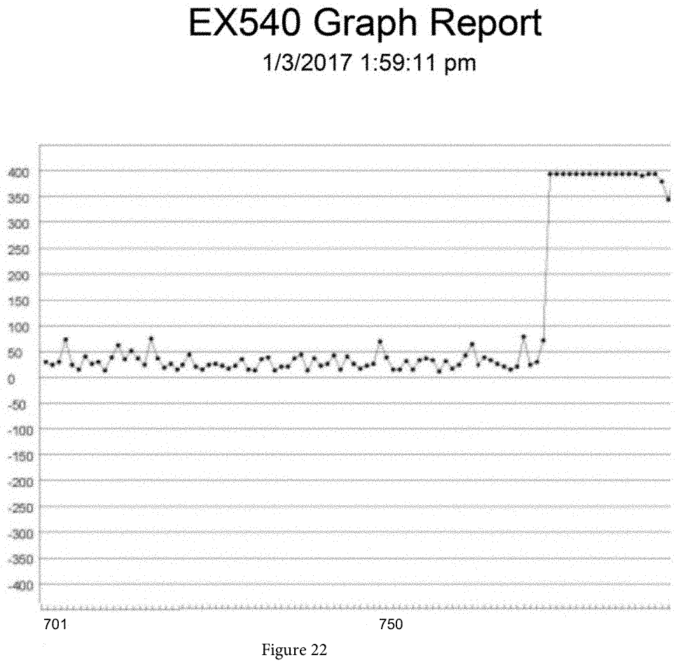

In FIGS. 21 and 22 the graph represents the millivolt differences when a saddle, made with the saddle tree disclosed herein, has been placed onto a horse at rest. There are regular and recurring oscillations in the milivolt readings, suggesting ongoing exchange of energy between the horse and the saddle tree.

Testing Example 2—Effects on Equine Physiological Systems

The saddles were used consistently on a daily basis for 1-2 hrs/day. Horses were housed in applicant's facilities with two of the horses recovered from Cushing's, and one having allergies. With continued use of the saddle as stated, the horses with Cushing's recovered and the allergies were eliminated.

The horses were under strict control and receive no medications, dietary supplements, or therapies, and no change in diet. All elements were tightly controlled, with the same riders, same amount of exercise, and the only variable being the saddle. A positive effect was seen on a broad spectrum of biological systems including nervous system, pain, cardiovascular, digestive and lymphatic systems. Multiple saddles were used in the testing, eliminating the possibility that the negative effects stemmed from a single poor fitting saddle.

The nervous system showed the first effect of exposure in that the normally agitated response to being saddled changed. Horses become measurably calmer as demonstrated by less movement, fidgeting, less tail ringing, biting, and pawing.

The next observed effect of the saddletree was pain. Back pain, common in horses, is checked by palpation along the horse's spine. During palpation horse flinches or pulls away or in extreme cases partially collapses. On the tested horses having a demonstratively sore back a normal saddle left on and at rest for 20-30 min exacerbates the back pain. The disclosed saddle, left on and at rest for 20-30 min completely or nearly completely alleviates the back pain.

The reduction in pain was also noted in how the horses moved during exercise. Leg stiffness due to join pain was eliminated and carriage improved.

Cardiovascular improvement, determined by heart rate, was vastly improved an is described in more detail below.

Digestive systems were improved evidenced in a virtually elimination of diarrhea, disappearance of allergies and improved coats.

The lymphatic system showed improvement through the lack of edema associated with horses their age.

Hydration suspected to be the primary cause of the majority of improvements. In the test subjects' hydration increased without extra water while recovery time after exercise was reduced.

Improved equine cardiovascular functioning.

Background

The following study of three saddles was undertaken based on expansion of an existing model used by the designer/manufacturer of the Tad Coffin saddles. Two saddle models were utilized, the A5 and the TC2, both close contact saddles designed for use in hunter/jumpers. In the data attached these saddles are referred to with the symbols TC. The third saddle was a Steuben, all -purpose saddle with a deeper seat, originally purchased for the fit of the tree, then classified by the manufacturer as a narrow tree.

Based on the designer's preliminary data on equine heart rate while wearing the TC saddles, a small study was made comparing resting heart rates between the two saddle types. The horse was ridden in one or the other saddle for a 30 minute period. This procedure was followed for a minimum of 5 consecutive days for each saddle.

Subject and Materials

Horse—15 year-old thoroughbred gelding; 17 hands, with typically prominent withers; show hunter; sound cardiovascular, respiratory and musculoskeletal systems prior to and throughout the experiment.

Observer/experimenter—Horse's regular rider, a retired equine veterinarian with background in physiology and pharmacology specific to anesthesia, trained and certified in animal chiropractic with several years of practical experience in this discipline with the horse.

Saddles:

Tad Coffin Performance Saddle A5 and TC2.

A Steuben all-purpose saddle with narrow tree width selected from a number of saddles for this particular horse. This saddle is the observer's normal, everyday saddle.

Girth—Professionals Choice nylon backed with neoprene panel, elasticized on both buckle ends (this horse's everyday girth)

Padding—Standard, contoured cotton quilt under pad and a Mattes fleece half pad (this horse's usual padding)

Experiment

Melissa Holland DVM performed the following testing at her barn and a full report is included below. Heart rate (HR) was measured by digital palpation of the left facial artery as it crosses the medial surface of the mandible. The study was begun each day in the horse's stall. The horse was not tied or cross tied at any time but allowed to move freely around the stall. The horse was groomed as usual in preparation for the day's ride. Heart rate was measured, noted as elapsed time zero (0) in the attached data, and then the saddle was applied and positioned as normal for this horse. The girth was applied so that a finger was easily slipped between the horse's rib cage and the girth. Heart rate was measured by a vet thereafter every 5 minutes and recorded for a total of 20 minutes. Heartrate was measured by placing a finger under the jaw bone and timing the heartrate with a stopwatch.

This experiment began by determining the effect on heart rate, if any, between either the Tad Coffin A5 or TC2 and the Steuben saddle. The Tad Coffin saddles produced a significant reduction in heart rate and this effect was prolonged. This suggests that repetitions of this study should provide that the order of saddle placement should be such that the Tad Coffin saddle is the second saddle tested. In other words, apply the non-Tad Coffin saddle first to reduce the possibility that the horse's resting heart rate will be inaccurate.

The horse was then ridden for a total of thirty (30) minutes. For the first 10 minutes, the horse was allowed to walk on a loose rein with contact gradually taken for the latter half of this warm up period. The last 20 minutes consisted of trotting and cantering. Occasionally low verticals were jumped. At the end of the 20 minutes, the rider brought the horse to a walk, dismounted immediately and took the first heart rate measurement. This point is noted as elapsed time 50 minutes in the data chart and table attached. At elapsed time 52 minutes, 55 minutes, 60 minutes, etc., the heart rate was measured. At approximately elapsed time 55 minutes, the saddle was removed. At approximately elapsed time 60 minutes, the horse was given a bath with tepid water to assist cooling (as usual post-work for this horse during warm weather). Heart rate was measured for a minimum of 60 minutes after the end of the horse's work (last measurement at elapsed time 110 minutes). These readings are reflected in the chart of FIG. 16.

Data was gathered over a period of five riding days for the Steuben saddle and eight (8) riding days for the Tad Coffin (TC) saddles. The median heart rate for each elapsed time period from 0 to 110 minutes was compared.

Results

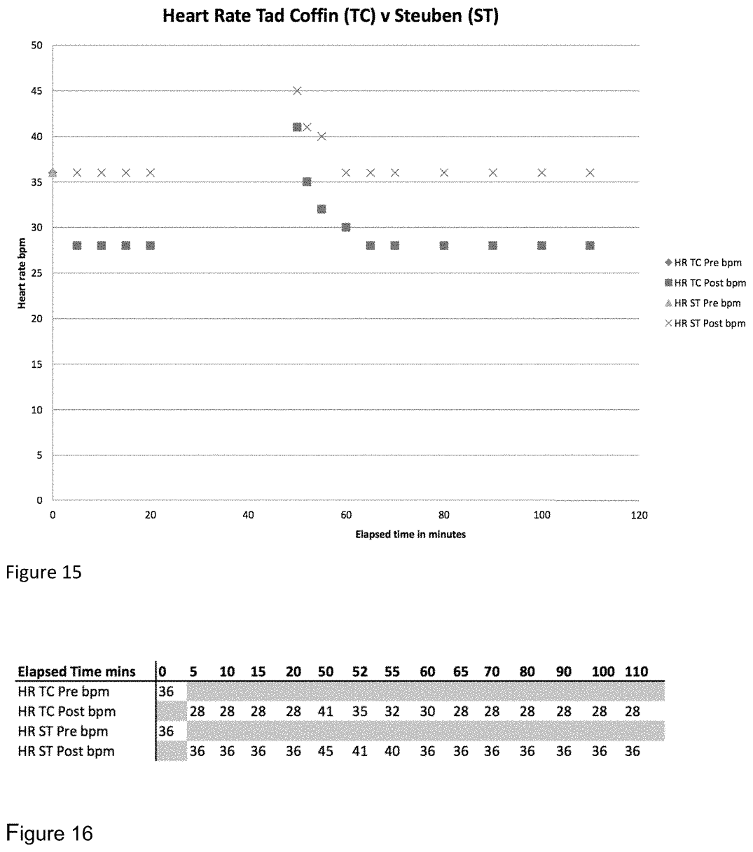

As can be seen in the table of FIG. 16 and graph of FIG. 15, the horse's median resting heart rate was consistently 36 bpm at time zero. The application of both the TC2 and the A5 saddles produced a significant reduction in heart rate. The heart rate dropped approximately 30% by the first reading at 5 minutes after saddle application and remained at this level throughout the 20 minute pre-ride period. The Steuben saddle produced no change in heart rate pre-ride.

Return to resting heart rate, 36 bpm for this horse, occurred on average, within 3 minutes post ride for the Tad Coffin saddles. For the Steuben saddle, 11.7 minutes was required for return to the horse's resting heart rate. Interestingly, the horse's heart rate continued to drop below the resting rate for the Tad Coffin saddles and consistently stabilized at the pre-ride-with-saddle-on rate of 28 bpm. The rate remained at this level for at least one hour (60 minutes) post ride. This phenomenon did not occur with the Steuben saddle as illustrated in the graph of FIG. 15.

Other Observations

The effect on the horse's heart rate recovery produced by variations in ambient temperature and humidity could not be isolated in this study. Nor could the influence of respiration be easily correlated with changes in heartrate. Respiratory rate post exercise was elevated, and even more so as humidity, in particular, climbed. These findings would be expected intuitively, but there was no consistent correlation with heart rate. Post exercise cooling was assisted by hosing with tepid, not cold, water. The application of water to the skin areas supplied by the great veins of the neck and legs and then to the entire skin surface is a common method used to speed cooling in horses post exercise as is application of circulating air, usually supplied with fans. Fans were not used for cooling in this study.

In general, the horse was alert during work, though he seemed almost sedated in the stall. This is a quiet individual under most circumstances, so this latter observation may have nothing to do with the effect of a saddle. Interesting side note: this horse does not love horse showing. He seemed much less ‘worried’ about the whole horse show experience after riding in the TC A5 each day.

Measurements and trends with the two Tad Coffin saddles were fundamentally similar. The TC2 was first evaluated. The rider returned to the Steuben and measured heart rate for 5 rides. Then measurements were made with the A5 saddle. Riding in and out of these saddles at these weekly intervals was subjectively interesting. The horse's way of going in the TC saddle seemed to ‘hang over’ into the time spent riding in the Steuben (say around 4 rides). Once returned to the second TC saddle, the A5, there was a period of about 2 days when the horse seemed to be ‘testing or experimenting’ with the saddle change. Again, on about ride number 4, he settled back into the TC frame and way of going (difficult to describe other than to say better balanced and more efficient self-carriage in all gaits; the sense that the rider must remember not to over-ride. Communication is more efficient?)

Suggestions for Further Study

The mechanism for the heart rate changes and differences in recovery to resting heart rate post exercise produced by the Tad Coffin saddles is unknown. The recovery to resting heart rate after exercise was significantly faster with the Tad Coffin saddles (median 3 minutes) than with the Steuben saddle (median 11.7 minutes). Even more curious is the initial drop in heart rate produced by the Coffin saddles pre-exercise and the persistent lower than resting heart rate eventually reached post-exercise.

Further monitoring of the post-exercise heart rate would be helpful. On two occasions where measurements were made, the heart rate remained at this level for at least two hours post exercise in the TC saddle. This effect was also evident at competition (horse show). The heart rate and heart rate recovery results were similar to those observed ‘at home’.

Levels of the stress hormone Cortisol will need to be tested as both heart rate and cortisol levels are affected by the balance between sympathetic and parasympathetic nervous system function. Sympathetic is the fight or flight system causing the heart rate to go up. Parasympathetic acts as a counterweight to the sympathetic system, causing the heart rate to come down. To test the rise or fall of cortisol, and the correspondence with the heart rate decrease noted above, levels of resting cortisol and heart rate in the horse in the pre-exercise condition need to be tested. After 20 minutes in the non-TC saddle the measurements would be repeated. These tests should be repeated and documented for several days. Then do the same experiment for the TC saddle. Baseline blood cortisol levels would have to be well documented in the study subjects such that any changes which accompanied the application of a saddle would be measurable without too much artifact. In order to minimize the effect of outside influence on the horse's cortisol levels, observers would most appropriately be the subject horse's regular rider and the horse should be in a familiar environment with regular routine.

Based on the results of the study described herein, other studies that would be of interest include:

A study comparing effect on heart rate by saddles where the saddle design/manufacturer is unknown to the observer and where the saddles are substantially similar in appearance

A study measuring heart rate while stimulating acupuncture points along the bladder meridian where saddles typically are positioned on the horse's back.

Summary

The effect on heart rate produced by saddles designed by Tad Coffin (TC) Performance Saddles and a Steuben saddle was measured in a fit, 15 year old thoroughbred gelding both at rest and after 30 minutes of exercise at the walk, trot and canter. The TC saddles produced a 30% reduction in heart rate at rest and a return to resting heart rate (36 bpm) post exercise in significantly less time—3 minutes for the TC saddles vs. 11.7 minutes for the Steuben saddle. Further reduction in heart rate below resting level post-exercise was produced by the TC saddles, but not by the Steuben saddle. Application of the Steuben saddle did not produce a reduction in heart rate at rest nor did this saddle produce a post-exercise reduction in heart rate below resting heart rate.

Notes on the Table and Chart

The above chart has a gap between elapsed time 20 minutes and 50 minutes. This gap corresponds to the 30 minutes of riding where measurements could not be taken. The first measurement is at the end of the ride. The total elapsed time at this point is 50 minutes.

The dot just above 35 on the heart rate axis is the resting heart rate pre saddle application with each saddle. This horse's resting heart rate of 36 bpm is consistent from day to day. The heart rate decrease to 28 bpm with the TC saddles is noted by the boxes. Heart rate in bpm for the Steuben measurements is represented by the X's.

Heart rate measurements for each time point in the table are the median rates in bpm for the 5 days spent riding in the Steuben saddle (ST) and 8 total days in either the TC2 or the A5.

Further studies on structures larger and smaller than a saddletree should be conducted to verify that identical results are produced. Further, the test subjects should be expanded. To date most studies have been on equines with only antidotal input from people. User input has been a noticed reduction in back pain, increase in hydration, muscle function. All of the antidotal input to date appears to be directed to an increase in hydration by providing the cells with a greater access to the balanced water.

Testing Example 4 Electromagnetic field in the saddle tree and its effect on water

The electromagnetic field of the saddle tree has a definitive effect on water. The hypothesis is that water exposed to the field is somehow restructured or reconfigured. This appears to be the case for intracellular and extracellular water. This would explain the improvements in hydration levels and the associated health benefits that have been observed with test horses consistently using the saddle.

While testing is insufficient at this point to definitively support this hypothesis there are previously cited studies that support the concept of restructured water and its benefits.

If a quantity of bottled or well water is placed alongside a saddle tree of the disclosed description and the saddle tree is then flex tested for symmetry, the presence of water has no impact on the manner in which the tree flexes. If the quantity of water is left within the saddle trees' electromagnetic field for approximately 30 minutes and the saddle tree is retested for symmetry, the side on which the water is positioned will be significantly stiffer and resistant to flex given the same force applied to it. The opposite side will flex normally. The hypothesis is that the exposure to the electromagnetic field has reconfigured the water structure. When placed on one side of the saddle tree, it seems to create a positive imbalance within the energy field. When placed either directly in front of or directly behind the saddle tree on the longitudinal axis, the symmetry is unaffected. This additionally supports the idea of the presence of an energy field.

In addition, the water has been widely observed to have a smoother texture and little to no aftertaste.

This same test can be conducted with fruits and vegetables with significant water content. If initially placed alongside the saddle tree and the saddle tree is flexed, it will flex symmetrically. If left in the electromagnetic field for 30 minutes and the saddle is retested for symmetry, the side on which the item is placed has an impact of the flexibility of the saddle tree consistent with that of the water mentioned above.

Water that has been treated with chemicals or that has had refined sugars or salts added will cause the saddle, upon initial presentation alongside the saddle tree, to flex asymmetrically. This asymmetry is characterized by a weakening of resistance when force is applied. When left in the electromagnetic field for approximately 30 minutes, the water will not be reconfigured and subsequent flex tests will continue to show a weakened response to force along the side on which it is placed. The hypothesis is that the added substances create a negative imbalance in the energy field.

Test Example 5. Changing the flex modulus of a structure by creating a positive or negative imbalance in the energy field.

There is a relationship between the flex modulus of a structure of the disclosed description and the positive or negative imbalance of the energy field. This relationship is not well understood but the patterns of response in test environments are completely consistent. Thus a structure can be manipulated to respond differently to forces acting upon it, depending on whether the field was balanced or not and if not then whether the imbalance was positive or negative.

Tests have demonstrated that certain substances cause a negative imbalance in the energy field. This weakening allows a greater flex modulus and increased mobility when forces act upon the structure. These items are typically ones that contained highly processed additives or chemicals. Examples of these items are certain adhesives, refined sugars and salts, food preservatives as well as chemicals and many common pharmaceuticals.

Testing has also demonstrated that certain items create a positive imbalance in the energy field and cause a stiffening or reduction of mobility. These substances are typically unrefined and naturally occurring. Examples include organic raw sugar, Himalayan salt and reconfigured water.

Utilizing this phenomena to create variations in a structures' response to forces acting upon it could prove quite useful. An example of this could be a pair of wings constructed according the disclosed description. Conceivably the manipulation of wing shape could be altered to create a change of course.

Restructrued water consumed as well as restructuring water within the body's cellular structure.

General Observations

In addition to the effect the electromagnetic wave on physiological systems, the EM structure can also influence the way other structures flex by strengthening or weakening electromagnetic field.

In order to determine additional information on the effect of an anomaly on the flex of a saddle, standard adhesive tape was used to provide the obstruction to the electromagnetic field.

In the initial tests a small length of tape, approximately 2″, was placed with an edge along the centerline of the saddle at which point the side of the saddle containing the tape did not flex as readily as the opposite side. Upon turning the tape 90 degrees, the other side of the saddle flexed.

To test the extent of the electromagnetic field a 3 inch piece of clear adhesive tape was placed on a small piece of wood, sticky side up. The tape was adhered to the wood by folding under the ends. When placed to be perpendicular to the saddle on one side, the opposing side flexes. The wood/tape combination were moved away from the saddle a couple inches at a time until there was no reaction as far as flex. It was found that the field extended five (5) to six (6) feed from the pommel; six (6) to eight (8) feet from the cantle and three (3) to four (4) feet from the sides.

Other potential uses for the EM structure.

Air plane wings containing an electromagnetic field generated as disclosed could have the flex affected, to change lift or course alteration.

A test method to determine whether an item or a food is harmful to humans and animals. For example placing a cell phone on one side of a testing EM structure to see if the flex modulus is affected.

A saddletree, prepared as described heretofore, has been cut to approximately the size of a bicycle seat to form an EM structure. The EM structure has been clamped to more easily measure the electromagnetic field. In this smaller structure, the sides do not have sufficient length to flex as with the saddletree. In order to test whether this configuration still produced an electromagnetic field a glass of water next to the EM structure and left for 20 min. The water was then put it next to the EM structure in the form of a saddletree at which time the flex modulus was affect and the side proximate the water would not flex. This was repeated with the glass of water being moved systematically further from the EM structure. The effect of the electromagnetic field of the EM structure was strongest close to the saddle and diminished as the water was moved further away. The effect was relatively consistent up until about 3 feet and rapidly diminished thereafter. The maximum effective distance was about 4 feet.

When the surface of the saddletree is asymmetrical, e.g., tape or other interference placed on the surface, there is a change in the readings. When the surface of the carbon fiber is unobstructed, symmetric induction occurs; however, once the symmetry is broken, induction will not occur.

The electromagnet field affects the flex modulus of the saddle. Without any anomaly or obstruction to the saddle, such as unevenness in the carbon fiber, the flex of the saddle is equal on both sides. Once there is an anomaly, the flex is unequal.

There is a marked decreased resistance to flex in the proximity of heavily processed foods, such as soda or beer. Further, depending on the degree of processing, these foods apparently do not absorb the electromagnetic energy and continue to “draw” from the EM structure, never increasing the resistance to flex. One test included Maca powder which should have increased the flex resistance but rather decreased the resistance. Further inspection of the product, in small print, disclosed that it contained a chemical known to cause birth defects.

Further, organic foods, even those with low or no water content affect the flex modulus. For example, Himalayan salt and organic sugar strengthen the flex modulus while iodized salt and refined sugar weaken the flex.

One possibility for this occurrence is that the energy created by the electromagnetic energy field seeks a current balance between the items within the field. Therefore, an item placed next to a saddletree generating a field would either draw from the generated field or support the field. Once an item has a current equal to the saddletree it reflects back the current, reinforcing the saddletree and making that side extremely difficult to flex. Movement of the item from one side to the other of the saddletree causes the flex modulus to reverse sides.

It is believed that the effects of the electromagnetic field directly taps into the biological system's pain relief response, directly affecting the mitochondria. Having an EM structure close to food and drink appears to enhance the water within, making it more absorbable and usable by the body possibly through the change of the hydrogen bonds. The probably restructuring of the water, based on research as noted above, seem to be having a direct effect on intracellular hydration.

The health benefits from exposure to low frequency, oscillating electromagnetic fields, as discussed heretofore, is known however there is little research into the exact nature of this energy. From the results seen, and discussed hereinafter, the electromagnetic field produced by the disclosed EM substrate are similar to those areas considered to be geomagnetic anomalies, such as Sedona, Ariz.

A beta saddle for testing purposes was given to Melissa Holland, DVM, DACVA for the purpose of using and reporting on the changes, if any, in her horse during the saddle's use. The following are insights from Dr. Holland during the test period:

“A User's Observations

After a year using the SmartRide technology in the form of the TC2 saddle, I have observed all of these effects. I have also observed reversions in some of these effects when the saddle is not functioning correctly.

Initially, I did some very basic research into changes in heart rate with the SmartRide saddle. Why? I noticed in the first few rides that the horse just didn't seem as hot, didn't sweat as much, and recovered more quickly post exercise. What could be easily measured with little equipment? Well, heart rate of course! If you can take a pulse, you could repeat my study. In brief, the saddle produced a 25 to 30% reduction in resting heart rate and a post-exercise heart rate recovery to this new resting rate in a shorter time than was seen with comparable saddles without the SmartRide technology (up to 50% faster recovery with the SmartRide saddle). The results of that study are available upon request. Please feel free to repeat it as well.

Faster recovery from exercise was not only measurable in heart rate recovery, but observable. In fact, this was the effect which started my ‘research’. Exercise induced sweating was less when ambient temperature and humidity were lower than 75 degrees F. and 60% respectively. Sweat dried more quickly. This resulted in reduced need to cool the horse with water hosing. For horses with poor hoof quality, applying less water to cool or remove sweat means less hoof wet/dry cycling , which is bad for hoof quality and water content.

I noticed right away, within a few rides, that the horse's attention to the work was improved. He was less distracted and less reactive, or over-reactive, to stimuli than when working in my other saddles. He paid attention. Is this a result of being more comfortable in this technology? I believe so. Remember those muscles of the topline. They are, with the spinal column in that region of the horse, literally the drive train. If those muscles are happy and functioning optimally with saddle and rider in place, the work of work should be easier and likely more efficient.

Consider the runner or the cyclist or the down-hill skier. The science that has gone into helping these human athletes to shape their stride and body movement to produce the most efficient movement is legion. The movement that covers the greatest distance with the least expenditure of energy and resistance is, by definition, the most efficient. We, the riders of the world, are way behind this technology curve. Why? Well, how do you teach a horse to be more efficient in his work? Ponder that. I think the best we can do is get out of his way as much as we can, while still giving direction, communicating what we want, and answering back when the horse responds. What if a saddle becomes something more than just a place to sit and drive the work? What if it becomes a tool that promotes efficiency of movement and that improves communication between horse and rider? Is this not, collectively, the definition of making the work of work more efficient? What stands in the way of making work more efficient? Resistance. I suspect that horses spend more energy than they should just to work around saddles that don't promote efficiency either because they are just not comfortable to wear, or they don't move with the horse and so impede efficient and forward motion. Maybe the observation of quicker recovery after exercise is partly explained by a horse that doesn't have to work as hard to answer the questions the rider is asking because he is not being impeded by a saddle that doesn't move with him.

Other Observations

Skin

I have a moderately itchy thoroughbred in the summertime. All year long, he is prone to skin disease—scratches mostly caused by the skin-resident bacteria Dermatophilus congolensis. It is a constant battle. Scrub it with medicated shampoo? Dry it out with astringents? Wave a voodoo doll over it? Nothing slows it. Let it rain more than a sprinkle and hives is the answer next morning. Whip out the anti-histamine. Delay the steroid as long as you can. You know this horse.

You may have one yourself. I have observed that this year has been different. There are the usual horse fly bites, but the ‘creepy crawly’ skin crud is pretty much gone. We pretty well understand that chronic inflammatory skin disease in horses has an immune system component. Some horses cope pretty well; some don't. Why? This bug is a resident skin bacterium. The horse's immune system ought to be able to keep it in its place.

I believe that the immune systems of all mammals are under assault all the time. Everything we eat and drink is grown in chemically treated and mineral-depleted soils. The rain that fills our aquafers, lakes, and streams is acidic and chemical-ridden. We are distracted and over-stimulated all day and half the night. We are functionally disconnected to the ground we tread. Except for maybe that last one, our horses are no different. Most don't get enough life with the herd. Most don't get enough turn-out, and they are lucky if there is a ‘weed’ in sight. Did you know that thistle is very high in calcium? Ever watch a horse graze? They eat dandelion if it's there. They eat thistle if it's there. Their water pretty much comes from the same sources as our water. They don't have the option of bottled water. They drink what we put in the bucket or the trough. It's no wonder the immune system is in overdrive. Much like people, the horse's immune ‘flare’ shows up in the skin, the respiratory tract and to a lesser extent, but no less important, the digestive tract. The skin is the thing we see every day. Does the itchy horse have an immune system that doesn't work as well or is it working in overdrive? Either way, it is not in balance and not working efficiently.

Is this improvement in immune function as evidenced by a middle aged horse that seems finally to be keeping the ubiquitous dermatophilus under control somehow related to the SmartRide-Rx technology? I don't know, but it's the only thing that's different.

Water Consumption

I have noticed that my horse's daily water consumption while in the stall seems to be less than last summer. Some days he hardly drinks. He's only in the stall about 6 or 7 hours during the hottest part of the day. But most days during that time, he gets his daily ride. And, it's been a hot and humid summer here in western Virginia. The horse does his work. He sweats and he cools off. We keep a gallon bucket in the wash stall for the cat. In the past, this horse would go to the wash stall for a bath and drain the cat's water bucket. Lately, he hasn't done that much if at all. He hardly drinks in the stall. I know he drinks from the trough in the field; I have watched him. The water comes from a well. It tastes good. I drink it myself. The buckets are dumped and cleaned every day. I′ve tasted the water in the bucket. It tastes fine. He is not dehydrated. He licks his slab of Redmond salt, but he does not drink a lot of water from that bucket. We used to give him two buckets; now he has one. He might drink 2 gallons in that 6 or 7 hour period. The grass is green and lush, just like last summer. The hay is good orchard grass just like all year. He's about as interested in it as he ever is during summer -not much because the grass is green and lush. Grain is the same. Supplements are the same. I′d be worried but the horse isn't telling me I should be. I′ve known him most of his life and I know his habits. SmartRide-Rx? I don't know, but it's the only thing that's different.

Feet

I ride a 17 hand thoroughbred with thoroughbred feet. I don't need to say much more. If you own one you get it. It's now July, and October needs to get here. But so far, only one lost shoe in the last six months. As always this time of year, the last 4 or 5 days before the blacksmith comes are nail biters, but so far so good. It's an improvement. Full disclosure, I feed a hoof supplement and I top dress feet with a sealer a couple of times a week. But the ravages of dew and rain-soaked grass and pounding on hard ground when it isn't raining always make for perilous shoe watching. Nothing new to this routine. SmartRide-Rx? I don't know, but it's the only thing that's different.

Muscles, Ligaments and Joints

I'll just start with a list of the stuff that I am always watching and waiting to blow. Hocks with DJD, old high suspensory ligament injury (minor, but twice), and some odd, isolated “splinter” the vet says is living just below the skin on the front of the lower cannon area of the right fore. Yep, she saw that on ultrasound. Magic! This last doesn't seem to bother the horse, but we watch it, wrap it to ride and ice it when we're done out of an abundance of caution, because we know swelling is an invitation for mischief, especially in a teen-aged horse. As for the ligament, so far, there is no new issue. Hock injections happen about once a year. We are coming up on the anniversary of that procedure. If we get through August with no injections, then I will be completely convinced that the only thing that's different is the only thing that will have mattered—SmartRide-Rx.

And Now for a Theory

As you can see my observations relative to the horse's physiology that might be affected by the saddle represent a diverse population of systems—cardiovascular, respiratory, immune, musculoskeletal, central and peripheral nervous systems, skin and hoof. I have read and researched for over a year to come up with some common denominator, some highly conserved mechanism that might explain these effects. In short, I didn't come up with a uniform theory. I dug into receptors and cell wall channels, cell wall pumps, neurotransmitters and mechano-baroreceptors. Nothing was ubiquitous enough or well-understood enough to explain such diverse effects. Mr. Coffin had suggested, from his similar observations, that what we were seeing might be explained by something as omnipresent and necessary, yet poorly understood, as water. This sent me down the rabbit hole of all the ways that water can be modulated, treated, ionized, re-structured, ordered and so on. In short, there is a lot of theory out there but not much real scientific evidence. That's just my opinion, but being a scientist by training, I am a sceptic.

True scientific cause and effect is a fleeting destination. But, I did come upon an intriguing theory postulated by Dr. Gerald H. Pollack, Professor of Bioengineering, University of Washington, Seattle. His interests range broadly from cardiac dynamics and electrophysiology to muscle contraction and cell biology. Dr. Pollack suggests, and backs it up with some pretty elegant research, that water has a fourth phase. This idea is not new. In fact, some version of it was postulated in the early 1900's. According to Pollack, this fourth phase of water behaves like a polymer gel. Dr. Pollack proposes that the interior of the cell, the cytoplasm, which is predominantly water, is also structured like a gel and has the same properties. The water molecules are structured or ordered in layers forming a matrix which makes water's positive charge more available to the proteins in the cell. Proteins are water loving because protein has a net negative charge. The proteins and the water constitute the gel.

So, back to water. Based on Pollack's theory, water has four phases—liquid, gas, solid, and gel. The gel phase of water is the state which lies somewhere between liquid and solid. In this state, water is ordered and has affinity for surfaces. This structure depends on the positive charge of the 2 hydrogen molecules and the negative charge of the one oxygen molecule and their ability to donate charge to nearby proteins. This gel phase is somewhat unstable—more stable than water, less stable than ice. In this state of moderate stability water can ‘donate’ a positive charge. Remember that the cell is packed with protein. Proteins do a very great percentage of the cell's work and are integral to energy production; neurotransmission; transport of solutes, nutrients, and cell waste; muscle contraction; cell division; etc. Proteins are negatively charged on the exterior. This negative charge makes protein highly hydrophilic (water loving). Cellular proteins are also highly structured. Think of the muscle cell (sarcomere), arguably the most organized of mammalian cells, built to do one job—contract—and then to relax. The proteins of the muscle sarcomere are almost rigidly organized as compared with synthetic gels which are typically built of tangled polymers with little apparent order. The protein of the muscle is predominantly of two types—myosin and actin. Both are highly water loving. In fact, actin is probably the most highly conserved protein in mammalian physiology and it is found in some form in every cell. This protein structure is of significant importance in how its association with ordered water can produce a response to stimuli which is both rapid and decisive. Under Dr. Pollack's theory, it is this protein water association which drives all of the basic cell functions.

The framework of the theory lies in the cell cytoplasm's similarity to the polymer gel. Both are built of cross-linked polymers; both contain ordered water; both exclude large solutes; both exhibit sizeable electrical potentials; and both have the ‘feel’ of a gel. If we can equate the character and function of the cell to that of a gel, then one can ask whether this can lead to a common paradigm for the cell's many actions.

The principle cell dynamics are secretion, production of action potentials which drive neurotransmission, transport, cell division, and contraction. According to Pollack, every one of these functions can be driven and recovery from the resulting action is promoted by how water and its various chemical configurations interact with the charged proteins in the cell by a process called phase transition. It's complicated and I don't pretend to be able to fully explain it. For the intrepid student refer to Dr. Pollack's excellent book—Cells, Gels and the Engines of Life.¹

The cell's interior, the cytoplasm, has a net negative charge. This charge is primarily due to the abundance of proteins within the cell. So-called ordered water, ie, the fourth phase of water, exists in the vicinity of these proteins, readily positioned to initiate the phase transition leading to a number of functions and then to reset the system to make ready for the next stimulus. Any number of stimuli may trigger the phase transition—change in pH, temperature, chemical or biochemical agents, salts, electrical fields, mechanical stress, electromagnetic radiation.”

How Does the Saddle Come Into Play?

Looking back at that list of stimuli given above which may produce a cellular phase transition, there are only two which might be relevant to the saddle—change in electrical field or electromagnetic radiation. The saddle is an external instrument resting on the horse's back. It cannot, in and of itself, produce a change in pH, temperature, chemicals or biochemical, salts or a mechanical stress which might produce the cellular responses outlined above.

The SmartRide-Rx saddle tree is composed of a polymer and carbon fiber, melded together in a patented process and then covered in padding and leather. It looks like most other saddles on the outside. This tree, however, is not doing ordinary things. Without doubt, it is designed to move with the horse. This alone should make the horse more comfortable, and therefore, more efficient in its work. However, how does this explain the other physiological effects observed—faster recovery from exercise, improved skin condition, reduction in muscle pain and fatigue, relaxation, improved attention to instruction? These effects originate in diverse physiologic systems—cardiovascular, pulmonary, integumentary, musculoskeletal, central and peripheral nervous—leading the observer to conclude that the effect, whatever it is, is not specific. What stimuli are left to consider? Only two remain—change in electrical field or electromagnetic radiation.

Mr. Coffin has determined that the saddle tree emits a field of energy and this field is approximately 9 cubic feet in area, that is, a three dimensional field above, below, in front of and behind the saddle. The field has a left and right hemisphere, and these merge in front and back. This field appears to generate a radiant energy in the infrared range as crudely detected with an adaptor for smart phone camera. See FIG. 2.

FIG. 2—Saddle Tree Photographed at Ambient Temperature with Infrared Lens Attachment

Based on Dr. Pollack's theories, we remember that one of the stimuli which triggers phase transitions in the mammalian cell was electromagnetic radiation (EMR). In the last decade, there is emerging evidence in the medical literature which suggests that EMR in the infrared range (just beyond the long wavelength red edge of visible light) promotes the production and function of ordered water (also commonly referred to as ordered water, concentrated water, nanostructured water, structured water). Externally applied EMR may selectively target the organization of this ordered water to effect biological functions. Existing data have not yet unequivocally proven the role of ordered water in modulation of cellular function, but research does show that such water can store charge and can later return it in the form of intracellular current flow with as much as 70% of the input charge being readily available. This charge in the ordered water is stable for days to weeks.² Dr. Pollack and others have demonstrated that radiant energy in the far infrared range (FIR) can order the water that possesses the structured arrangement which promote these cell functions by phase transition.³ In 2016, a group of researchers proposed that EMR in the infrared range can produce light-water interactions stimulating the cell to generate high energy molecules such as adenosine triphosphate (ATP) which supply energy for cellular functions including metabolism and signaling.

We are surrounded by infrared radiation (IR). Remember, the sun generates this energy. We cannot see it, except with a device which detects it, because IR lies outside the range of visible light on the electromagnetic spectrum. See FIG. 3.

FIG. 3

The source of energy in both the visible and infrared (IR) portions of the EM spectrum is the sun. Objects absorb and reflect that infrared energy. A review of the available literature indicates that far infrared energy (FIR) has the greatest likelihood of producing the diffuse biological effects detailed above. Medical researchers have developed instrumentation to deliver concentrated IR energy from the near IR (NIR) to the far IR (FIR) range such that tissues may absorb and/or reflect IR energy. These instruments are often as simple as infrared heat lamps emitting in the NIR range and as sophisticated as specialized far infrared lamps which utilize ceramic emitting panels that remain cool to the touch. The infrared energy emitted by these panels would be absorbed by the target tissues. One comparative study showed that FIR therapy reduced symptoms of exercise-induced muscle damage in athletes after a simulated trail running race.⁴ A study of patients with rheumatoid arthritis and ankylosing spondylitis showed a reduction in pain, stiffness, and fatigue during infrared sauna therapy.⁵

Conversely, the body itself is a reflector of absorbed, ambient infrared energy. FIR emitting materials such as the boron-silicate mineral, tourmaline, when milled into fine powders, can be incorporated into fibers to make fabrics which can be applied to various body areas. Infrared energy being reflected off of the human body is believed to be transferred to these materials which are acting as “perfect absorbers” which then emit FIR back to the body. Since this is a thermodynamically neutral process, there is little risk of excessive heat production which might cause tissue damage. FIR emitting ceramics have been shown to delay the onset of fatigue in a simulated skeletal muscle contraction model⁶, to reduce foot pain and inflammation in human patients with diabetic neuropathy⁷, and to suppress proliferation of some types of cancer cells.⁸

What does all this have to do with a saddle tree? I postulate that the materials in this tree, as constructed, are either emitting EM radiation in the FIR range, and/or are acting as so-called “perfect absorbers” of the FIR energy being reflected by the horse's and the rider's body. Whichever the mechanism, the resulting energy is sufficient to slightly perturb the ordered water matrix along the cell's proteins such that phase transition can occur. These phase transitions then lead to the various mechanisms that produce the multitude of mitochondrial and cell functions that led off this discussion.⁹ Phase transition may be a common mechanism among many cellular organelles. It is simple—on or off, and it is powerful in its effectiveness. A subtle environmental change triggers an immediate and massive response.

Cellular protein and the ordered water which surrounds these proteins and the surfaces of other cellular organelles require energy to maintain order. In fact, some of the cell's energy is stored in the form of order. Consider the muscle cell. Initially, water is ordered around thick and thin protein filaments. These filaments lie in their extended, high potential energy states. Following some stimulus, order gives way to disorder. As filaments contract and surrounding water is released into disorder, potential energy is given off in the form of mechanical work and heat. Then the system must be re-primed. The filaments need to be restored to their lengthened state and water needs to be restructured. Re-ordering requires energy. Where does this energy come from and how is it used to create structural order? Enter adenosine triphosphate (ATP).

ATP has very high affinity for proteins and is highly charged. Due to this charge, ATP is able to shift the electron cloud of adjacent atoms, which, in turn, shifts the cloud of a subsequent atom and so on. Through this sequential action, binding of ATP produces protein extension which is reinforced by water structuring. Because ATP binding is so tight, the energy stored and released is substantial. It is this protein bound ATP, ordered water, energy building cellular machine which keeps the extended protein ready to respond to whatever stimulus will generate it's specific action.

ATP probably initially arose from the primordial ooze. Here we go back to the proverbial big bang theory of evolution. Under the prevailing atmospheric conditions, and under the influence of the sun's energy, experiments have shown that ATP could be created in much the same way that amino acids and DNA could be created. Unfortunately for that first assembled gel of life, atmospheric oxygen was still in short supply. The first cells had to rely on very low-yield anaerobic (without oxygen) processes to generate ATP. Basically there was probably just enough ATP to keep this emerging biologic system alive.

The landscape changed with the arrival of atmospheric oxygen. As primitive cells began to release oxygen from photosynthesis to the atmosphere and as volcanic activity subsided, reductive gasses began to be replaced by oxygen. Metabolism could use that oxygen. Certain unicellular organisms such as bacteria began to utilize oxygen to produce ATP. ATP is produced on the bacterial surface. That surface, is, therefore, heavily negatively charged because ATP is negatively charged. Turns out, at some point on the evolutionary curve, one of those primitive bacterial cells which had learned to employ the new oxygen-based metabolic system to produce energy in the form of ATP invaded a larger cell that had not. This was the first cellular organelle. We now know it as mitochondria, because that invading bacterium stayed, and it knew how to make ATP.¹⁰

The invader's high negative surface charge in the form of ATP could order the host cell's water and water ordering could then induce extension of nearby host cell proteins. As long as oxygen could diffuse through the host cell to fuel continuous ATP production, the invader could be exploited to produce potential energy for the host cell. This scenario is supported by nuclear magnetic resonance studies which demonstrate water structuring inside and outside mitochondria.¹¹ That single cell bacterium now evolved to single-mindedly produce energy for the host cell in the form of ATP. The rate of energy production was further enhanced by the development of in-foldings in the mitochondrial wall called cristae, and these enlarged the surface area for the production of ATP. Mitochondria multiplied in number and became positioned adjacent to the most aggressive consumers of energy—proteins. With the mitochondria now efficiently producing sufficient supplies of energy, the host cell could turn its attention to specialization.

Once bound to intracellular protein, ATP's strong negative charge lengthens or extends the protein. This lengthened protein orients its negative charge to the outside and water in the vicinity is ordered. The resultant order holds energy. The system is now primed to do work. As some stimulus comes along and discharge of the potential energy of this order occurs, ATP is split into adenosine diphosphate (ADP) and phosphorus. ADP is less negatively charged than ATP, so it is forced off the protein and moves down its concentration gradient to the nearby mitochondria so that more ATP can be produced. Note here that this cycling of ATP is also the cycling of charge. With each ATP that binds to protein, the protein gains negative charge. Water in the vicinity is ordered according to this negative charge. This negative charge on the protein builds but not indefinitely. The protein must eventually discharge and this occurs when ATP breaks down to ADP and phosphorus. The protein is returned to its discharged state and the cycling of ATP and protein recharging follows. So, the protein is charged to accumulate energy and discharged as some action such as contraction, secretion, ion transfer, etc. is produced. So the cell is an energy machine. It prepares for action by building and maintaining order and then releasing energy as some stimulus discharges or disorders the system. This ATP cycling is fundamentally what drives the phase transition—a newly proposed model of cell organelle function.¹²

The various forms of phase transition have a common component—cooperation. Transitions are not singular molecular events, but rather global actions. Once a critical threshold has been reached, the transition occurs instantly and cooperatively among the components structures. Remember the cell is full of proteins and ordered water. In fact, any cell structure which has predominantly negative charge on its surface can serve to order water. The cell membrane itself orders water because the membrane has a net negative charge. Mitochondrial surfaces are highly negative, and ordered water has been demonstrated on mitochondrial membranes.¹³ This interaction between negatively charged surfaces and intracellular water literally build the gel of the cell's interior and holds it together. The interaction between negatively charged proteins, driven mostly by ATP, orders water and creates energy.

The phase transition has a variety of transition forms. This variety reflects the diversity of tasks a cell must perform—permeability change, ion transport, solute expulsion, shape change, force generation, strand shortening, etc. Condensed energy—loaded vesicles are ideal for secretion; helix-coil transitions work well for contraction against loads; condensation from extended coils produce large scale length changes ideal for transport and cell migration. Diverse types of transition meet diverse functional needs of the cell such as respiration, neuronal transmission, toxin and metabolite elimination, contraction, nutrient uptake and metabolism, etc. As such, the phase transition is a simple and powerful process Coffin Page 47 triggered by small environmental shifts in pH, temperature, pressure, ion content, mechanical stress, electromagnetic radiation, etc.

The theory postulated here is that this small environmental shift is produced by far infrared (FIR) energy produced by the materials used to construct the SmartRide-Rx™ saddle tree. The cycle is as follows:

Process 1

FIR→↑ATP production→protein binding displacing ADP→protein extension, high negative charge→water ordering=energy=ready for phase transition to perform whatever the cell needs to do instantaneously

Then, Process 2

ADP+P returns down gradient to mitochondria→ATP production leading to Process 2, and so on . . . .

If the saddle material is emitting FIR energy then any cell within this field should be more efficient in its function due to augmented mitochondrial respiration, ie, ATP production. Mitochondrial membranes are believed to order water. IR therapy has been postulated to cause small perturbations in mitochondrial membrane structure by way of nearby intracellular Ca²⁺ which is theorized to initiate the contraction of proteins to which ATP is bound and around which water is ordered. The second common feature of phase transition is the involvement of divalent cations of which Ca²⁺ is by far the most prevalent. Calcium condenses the protein-water matrix and squeezes out the water. The protein contracts and stored energy is released. Work having been accomplished, ATP is regenerated and binds to the protein lengthening it so water can be re-ordered and order/energy is restored. The structure is ready to go again.

With respect to this theory, the most useful question to be answered is the characteristic(s) of the wavelengths being emitted or reflected by the SmartRide-Rx tree. At the cellular level, the underlying biophysical mechanisms of the interaction of electromagnetic radiation with living cells can be understood in terms of altered cell membrane potentials and altered mitochondrial metabolism, ie, ATP production.¹⁴ There is more work to be done. Stay tuned.

Recommendations for Use in the Horse

Based on my use of the saddle and observations of its effects and observations made by the manufacturer in field trials over many years and rides, I have the following recommendations for use of the SmartRide-Rx saddle for the typical horse in work.

Application

I tack up my horse in this saddle every day for a minimum of 30 minutes. I utilize the pad or pads which work best for my horse's anatomy, determined by trial and error and generally following the directions of the manufacturer. My horse is loose in the stall, free to eat at will. Usually by the 10 minute mark, he ‘naps’ as evidenced by leaving hay, lowering the head and drooping of the eyelids and often lower lip. These effects are similar to those which are often seen with acupuncture treatment or massage. Heart rate lowers to about 70% of normal resting rate. At the end of this period, which I find may be reduced to 20 minutes if necessary, I finish tacking up and proceed to ride if it's a riding day. These observations are consistent enough that if one does not see these effects and there are no other distractions which may be affecting the horse's demeanor or comfort (like a missing pasture buddy or delivery of a load of hay, etc.) one should probably give the manufacturer a call.

Interestingly, I have not found that the ‘sleepiness’ which occurs as the horse is standing around in the saddle continues once we get to work. If you have ever ridden a horse that has been tranquilized, you know what it feels like—sort of lazy off your leg and dull. I imagine it would be comparable to how humans feel after taking an anti-histamine or valium—sleepy and slow. The horse at work in this saddle is alert and ready to work, not at all dull.

Even on days when I can't ride, and winter time is full of those, I try to put this saddle on my horse for at least 30 minutes a day. My observation has been that I can go for days without riding, or get a ride in every other day if the weather cooperates, and my horse doesn't seem to lose fitness. I have talked with other users of this saddle who report the same impression. If real, this effect is helpful in all horses, but should be especially beneficial for the young horse just learning and the older horse with known musculoskeletal problems such as chronic degenerative joint disease or old tendon/ligament injuries.

Conclusions on My Year in the SmartRide-Rx Technology

I think my horse is healthier. In general, he is calmer. He's pretty calm to begin with, so the casual observer might not notice much difference. But I have had this horse for 14 years and I know the difference.

I find that my horse is less distracted in his ring work and less of a spook when hacking out either alone or in company. This saddle makes me feel more secure and spooks on the horse's part are less worrisome to me. This was a real surprise since I rode for years in a rather deep-seated, all-purpose saddle chosen because it should have made me feel more secure. Go figure! Let's face it, if the horse finds something scary to jump at, he's also ‘listening’ for the rider's reaction. If I am not worried by his reaction because I am more secure, the horse's spook matters less to me. I can ignore it and ride on and the horse, sensing no bother on my part, just decides there's nothing to see here and moves on. I would imagine that the act of jumping away from something deemed scary will abruptly put the horse's back up against the saddle, any saddle. If there's no resulting physical discomfort and the rider doesn't move or overreact, then the horse is free to just move on. In all phases of riding, I would describe the feeling that the rider achieves is one of “being in the saddle instead of on it”. I will note here that if the saddle is not doing its work effectively, one of the signs can be a spookier or more distracted horse. If you notice this change, call the manufacturer. DO NOT HESITATE! CALL!

At first use of the saddle, I found that my horse went through about 3 rides where he experienced what I would describe as questioning, almost as if to say, ‘What is this new thing?’ I call what I noticed a testing stretching. The rider feels the horse sort of tentatively stretch out through the back. And, I would imagine, not finding resistance or a ‘hard stop’ coming from that thing he is wearing, he lifts his back up into this saddle instead of meeting resistance with resistance. That is the only way I can describe the feeling. By about day 3, the back is coming up just about as soon as you walk off. The stride is lengthening and the horse's attitude is softer. He has learned that resistance is not needed. He doesn't have to adjust to find a comfortable place to be. I noticed right away that upward and downward transitions became smoother and more ‘through the back’.

As to jumping, the horse went forward more readily to the jumps, felt rounder over the jumps, and felt softer both on take-off and landing, coming right back after landing—just less resistant in all aspects than in my previous saddle. In general, I find that the horse ‘hears’ my instruction and I can feel him ‘answer’ sooner. This has really forced me not to ‘over-ride’ the jumps and to feel that I can wait for the ‘fence to come to me’. I think this is just the result of being better able to maintain a consistent stride because the horse is hearing and answering more quickly and efficiently. I imagine this would apply to all riding disciplines.

In my experience, if your horse is getting regular massage, chiropractic adjustment, acupuncture treatment, joint supplements or injections, etc., those treatments should not be discontinued, particularly in older horses with known orthopedic challenges. You may find, as I have, that your horse does not need these treatments as frequently. Horses that work hard, and particularly those that travel and/or horseshow frequently, will still benefit from these therapies.

This equipment is not tolerant of extremely cold temperatures. If your tack room is not heated in winter and the temperatures in your barn are below freezing, it's probably best not to store the saddle in the barn or in a horse trailer. Consult the manufacturer for recommendations. You have invested not only in advanced technology for communicating with the horse, if my theory is correct, you have also given your horse better overall health. Could it be as simple as just improving the production of the cell's energy? Why not? Don't forget good old ATP, ordered water, and the phase transition. Nature loves simplicity.

Treat this saddle as you would that smart phone appendage. You will be rewarded with a more comfortable horse, happier and more responsive in his work. And, that horse is just more fun to ride. Happy trails!”

Broad Scope of the Invention

While illustrative embodiments of the invention have been described herein, the present invention is not limited to the various preferred embodiments described herein, but includes any and all embodiments having equivalent elements, modifications, omissions, combinations (e.g., of aspects across various embodiments), adaptations and/or alterations as would be appreciated by those in the art based on the present disclosure. The limitations in the claims (e.g., including that to be later added) are to be interpreted broadly based on the language employed in the claims and not limited to examples described in the present specification or during the prosecution of the application, which examples are to be construed as non-exclusive. For example, in the present disclosure, the term “preferably” is non-exclusive and means “preferably, but not limited to.” In this disclosure and during the prosecution of this application, means-plus-function or step-plus-function limitations will only be employed where for a specific claim limitation all of the following conditions are present in that limitation: a) “means for” or “step for” is expressly recited; b) a corresponding function is expressly recited; and c) structure, material or acts that support that structure are not recited. In this disclosure and during the prosecution of this application, the terminology “present invention” or “invention” may be used as a reference to one or more aspect within the present disclosure. The language of the present invention or inventions should not be improperly interpreted as an identification of criticality, should not be improperly interpreted as applying across all aspects or embodiments (i.e., it should be understood that the present invention has a number of aspects and embodiments), and should not be improperly interpreted as limiting the scope of the application or claims. In this disclosure and during the prosecution of this application, the terminology “embodiment” can be used to describe any aspect, feature, process or step, any combination thereof, and/or any portion thereof, etc. In some examples, various embodiments may include overlapping features. In this disclosure, the following abbreviated terminology may be employed: “e.g.” which means “for example.”

What is claimed is:

1. An electromagnetic (EM) structure to emit a low frequency oscillating electromagnetic energy field comprising:
a nonpolar substrate, said substrate changing from nonpolar to polar upon application of direct heat to bring said substrate to a first predetermined temperature,
carbon fiber, said carbon fiber being applied to said substrate prior to reaching a second predetermined temperature, and
epoxy, said epoxy adhering said carbon fiber to said substrate.

2. The EM structure of claim 1 wherein said first predetermined temperature is between 160 and 180 degrees F.

3. The EM structure of claim 1 wherein said carbon fiber is soaked in said epoxy to saturation and excess epoxy removed prior to placement on said substrate.

4. The EM structure of claim 1 wherein said second predetermined temperature is greater than 100 degrees F.

5. The EM structure of claim 1 wherein said second predetermined temperature is greater than 50% of said first predetermined temperature.

6. The EM structure of claim 1 wherein said substrate is sanded in a crosshatch pattern and then washed and dried prior to application of said direct heat.

7. The EM structure of claim 1 wherein said direct heat is a propane torch at a distance to enable a blue portion of a flame to contact said substrate.

8. The EM structure of claim 1 wherein said epoxy is a mixture of soft epoxy and hard epoxy.

9. The EM structure of claim 1 wherein said substrate is Kydex.

10. The EM structure of claim 1 wherein said EM structure has opposing flexible sides, each of said opposing flexible sides having an equal flex modulus.

11. The EM structure of claim 10 wherein said flex modulus of each of said opposing flexible sides is increased or decreased depending upon a material placed proximate one of said opposing flexible sides.

12. An EM structure for the emission of low frequency oscillating electromagnetic energy field comprising:
a nonpolar Kydex substrate, said substrate changing from nonpolar to polar upon application of direct heat to bring said substrate to a temperature of between 160 and 180 degrees F.,
epoxy, said epoxy being a mixture of hard epoxy and soft epoxy,
carbon fiber, said carbon fiber being applied soaked in said epoxy, excess epoxy removed and applied to said substrate prior to reaching a temperature no less than 80 degrees F.

13. The EM structure of claim 12 wherein said substrate is sanded in a crosshatch pattern and then washed and dried prior to application of said direct heat.

14. The EM structure of claim 12 wherein said direct heat is a propane torch at a distance to enable a blue portion of a flame to contact said substrate.

15. The method of creating an EM structure for the emission of electromagnetic currents comprising the steps of:
a. Thermoforming a substrate into a predetermined configuration,
b. Sanding said substrate in a cross hatched pattern,
c. Washing said substrate,
d. Drying said substrate,
e. Flaming said substrate to bring a temperature to about 160 to 180 degrees F.,
f. Soaking carbon fiber in an epoxy mixture of hard epoxy and soft epoxy
g. Removing excess epoxy mixture from said carbon fiber
h. Apply said carbon fiber to said substrate
i. Drying said substrate carbon fiber structure to form an EM structure;
wherein said EM structure emits a low frequency, oscillating electromagnetic energy field surrounding said EM structure.

16. The method of claim 15 further comprising the step of forming said EM structure to have opposing flexible sides, each of said opposing flexible sides having an equal flex modulus.

17. The method of claim 16 further comprising the step wherein placing a material proximate one of said opposing flexible sides affects said flex modulus of said one of said opposing sides.

18. The method of claim 16 wherein said material is a first material containing water and is unprocessed and said flex modulus of said one of said opposing flexible sides is increased making said one of said opposing flexible sides more difficult to flex.

19. The method of claim 16 wherein said material is a second material containing water and is processed and said flex modulus of said one of said opposing flexible sides is decreased making said one of said opposing flexible sides easier to flex.

20. The method of claim 16 wherein said material is a third material containing water and is unprocessed and said flex modulus of said one of said opposing flexible sides is unaffected for a predetermined period of time and after said predetermined period of time said one of said opposing flexible sides is more difficult to flex.
